(19) United States
(12) Patent Application Publication
(10) Pub. No.: US 2025/0237641 A1
(43) Pub. Date: Jul. 24, 2025

(54) SYSTEMS AND METHODS FOR CHARACTERIZING PATHOPHYSIOLOGY

(71) Applicant: University of Washington, Seattle, WA (US)

(72) Inventors: Benjamin FREEDMAN, Seattle, WA (US); Louisa HELMS, Seattle, WA (US)

(73) Assignee: University of Washington, Seattle, WA (US)

(21) Appl. No.: 18/699,527

(22) PCT Filed: Oct. 7, 2022
(86) PCT No.: PCT/US2022/077761

§ 371 Date: Apr. 8, 2024

Related U.S. Application Data
(60) Provisional application No. 63/253,797, filed on Oct. 8, 2021.

(51) Int. Cl. G01N 33/50; A61K 48/00; A61P 13/12; C12N 5/071; C12N 9/48

(52) U.S. Cl. CPC G01N 33/5088; A61K 48/0008; A61P 13/12; C12N 5/0686; C12N 5/0697; C12N 9/485; C12Y 304/17023; C12N 2513/00; C12N 2770/20031; G01N 2800/34

(57) ABSTRACT

Systems and methods for culture of human kidney organoids as a model system for characterizing kidney pathologies and implementing therapeutics for conditions that affect the kidney. A model system for characterization of a pathophysiology includes a culture of human kidney organoids and an organoid culture medium. The organoids may include one or more genome edits as part of an approach for studying genetic factors involved with a pathological process. The culture may include one or more other factors, such as pathological agents and/or anti-pathological agents, for development and evaluation of therapeutics. The approaches may be used for implementation of compositions and methods for virally transducing the kidney or a subset of cells or cell types thereof.

### CROSS-REFERENCE(S) TO RELATED APPLICATION(S)

This application claims the benefit of U.S. Provisional Application No. 63/253,797 filed on 8 Oct. 2021.

### STATEMENT OF GOVERNMENT LICENSE RIGHTS

This invention was made with Government support under Grant No. W81XWH-21-10007, awarded by the Department of Defense, and Grant Nos. R01 DK117914, U01 DK127553, and UG3 TR002158, awarded by the National Institutes of Health. The Government has certain rights in the invention.

### BACKGROUND

Virally targeting the kidney is a challenging task for many viral vectors widely used in gene delivery strategies, including lentiviral vectors, adenoviral vectors, and adeno-associated viral (AAV) vectors. While infection of human kidney cells with severe acute respiratory syndrome coronavirus 2 (SARS-CoV-2) virus has occurred, it is unknown which of the kidney cell types would be effectively targeted by a SARS-CoV-2 viral vector, and it is not confirmed which is the primary viral entry pathway for SARS-CoV-2 infection. Characterization of entry of SARS-CoV-2 into the cell, and determination of which cell types enable this entry and how it occurs, would have significance for gene therapies and viral vectors that target the kidney, and specific cell types of the kidney, as part of clinical and non-clinical research and development. This would also enable modification of cells and cell types that are not tractable with existing viral vector technologies to become susceptible to infection with SARS-CoV-2 based viral vectors. There is a need for a reliable and accurate model system for studying human kidney pathologies, including SARS-CoV-2 as well as acute kidney injury (AKI), chronic kidney disease (CKD), polycystic kidney disease (PKD), and others. Such a system would also enable novel therapies for use in humans.

Infection with SARS-CoV-2 virus causes COVID-19. SARS-CoV-2 is a positive-sense, single-stranded RNA virus that utilizes an RNA dependent RNA polymerase carrying a high mutation rate, up to one million times higher than its hosts' DNA polymerase. Higher mutation rates correlate with enhanced virulence of emerging viral strains and is suggested to produce SARS-CoV-2 viral variants with enhanced infectivity. Patients with COVID-19 suffer from several symptoms, including respiratory distress, but they also exhibit systemic symptoms that involve the kidneys, similar to previous SARS-CoV and MERS-CoV outbreaks. It is unknown whether SARS-CoV-2 variants of concern (VOC) have different tropism to extra-pulmonary organs, such as the kidney.

The emergence of SARS-CoV-2 has sparked the rapid development of novel therapeutics aimed to block viral infection and replication. For example, the nucleotide analogue prodrug remdesivir was granted emergency use authorization (EUA) for the treatment of COVID-19 in May 2020 for its ability to inhibit viral RNA-dependent RNA polymerase. While some studies have shown that remdesivir treatment in AKI and CKD patients is tolerated well, the active metabolite of remdesivir is eliminated by the kidneys and has been reported to increase chances of developing AKI in remdesivir-treated patients. Accordingly, it is unknown whether remdesivir and other therapies may have unintended or detrimental effects at certain dosages, in certain combinations with other treatments, or when used as part of certain treatment regimens.

Efforts to better understand SARS-CoV-2 infection rely on human cellular and organoid model systems, which have played a valuable role in understanding SARS-CoV-2 infection mechanisms, interactions with key target organs, and the efficacy of COVID-19 therapeutics. The RNA genome of SARS-CoV-2 encodes three membrane proteins: the spike protein, which binds the cell-surface receptor to mediate virus entry; the membrane protein, which contributes to virus assembly and budding; and the envelope protein E. The SARS-CoV-2 spike protein (SARS-CoV-2 S) plays a key role in cell receptor recognition and cell membrane fusion processes, and is composed of two subunits, S1 and S2. The S1 subunit contains a receptor-binding domain that recognizes and binds to the host receptor angiotensin-converting enzyme 2, while the S2 subunit mediates viral cell membrane fusion by forming a six-helical bundle via the two-heptad repeat domain. The SARS-CoV-2 envelope protein (SARS-CoV-2 E) forms a homopentameric cation channel that is important for virus pathogenicity. Despite knowledge of mechanisms of SARS-CoV-2 pathogenicity for specific tissues, such as lung epithelium, it is unknown whether and how the virus infects cell and cell types of other tissues, such as kidney tissues.

Kidney organoids are segmented structures that resemble primitive nephrons, which can be differentiated in vitro from human pluripotent stem cells, including induced pluripotent stem (iPS) cells and embryonic stem(ES) cells. Human models are particularly valuable because mice are not generally susceptible to SARS-CoV-2 without adaptation to mouse angiotensin-converting enzyme 2 (Ace2). Specifically, while it is known that SARS-CoV-2 can infect kidney organoid cultures, a property that has been leveraged to test candidate therapeutics, such organoids contain about 16 different cell types, and it is unknown which of these specific cell types are infected by SARS-CoV-2, or how they are infected.

In addition, PKD is the most common genetic cause of CKD and is a known risk factor for developing severe COVID-19. In PKD, expansive cysts form from tubular epithelial cells. The PKD cysts can grow very large over time and can contribute to kidney damage and dysfunction. Whether and how PKD cysts are susceptible to SARS-CoV-2 infection are unknown and a better understanding of this may help explain the association of PKD with increased risk of development of severe COVID-19 as well as enable the development of novel gene therapy methods for targeting these structures.

Accordingly, there is a need for an improved human kidney model system for characterization of a pathogenesis for development of antiviral therapies and gene therapy compositions and methods that target the kidney or specific kidney cell types. The present disclosure addresses these long-felt and unmet needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In various aspects, the disclosure provides systems and methods for characterizing SARS-CoV-2 infection of human kidney organoids by using novel fluorescence-reporter SARS-CoV-2 variants. The approaches can involve determination of co-localization of SARS-CoV-2 infection with markers of apoptosis, which informs whether infection can produce direct cytotoxic effects that resemble AKI. The disclosure also provides approaches to compare clinical cohorts, study viral variants of concern, and screen candidate therapeutics related to kidney disease for safety and efficacy. The disclosed utilization of organoids that are genome edited also enables the determination of mechanisms of viral infection and assessment of the impact of pre-existing disease states on the severity of disease states that result from viral infection.

The disclosure provides a method for culturing a human kidney organoid as a model system for characterization of a pathogenesis, the method comprising: generating a culture that comprises the human kidney organoid and a human kidney organoid maintenance medium for the culturing of the human kidney organoid; contacting the culture with a viral preparation that comprises a severe acute respiratory syndrome coronavirus 2 (SARS-CoV-2) virus or a variant or component thereof; and determining whether one or more cells or cell types of the human kidney organoid are infected by the viral preparation and produce a SARS-CoV-2 infected human kidney organoid.

The disclosure also provides a model system for characterization of a pathogenesis, the model system comprising: a SARS-CoV-2 infected human kidney organoid; and a human kidney organoid maintenance medium for culture of the SARS-CoV-2 infected human kidney organoid.

The disclosure also provides a method for characterizing a pathophysiology, the method comprising: culturing a culture that comprises a SARS-CoV-2 infected human kidney organoid and a human kidney organoid maintenance medium for maintenance of the human kidney organoid; contacting the culture with a pathological agent; and characterizing a response of the culture to the pathological agent.

One or more cells or cell types of the human kidney organoid may be angiotensin converting enzyme 2 (ACE2) positive. In at least some embodiments, the method for culturing the human kidney organoid can further comprise modulating an ACE2 gene to produce one or more ACE2 modulated cells or cell types of the organoid and determining whether ACE2 gene modulation affects infection by SARS-CoV-2. In instances where the ACE2 gene is negatively modulated, one or both alleles can be genetically knocked out to produce one or more ACE2 negative (e.g., ACE2^{-/-}) cells or cell types of the organoid.

The human kidney organoid can comprise one or more genome edited cells comprising one or more genomic edits for the characterization, and the characterization can comprise a determination of whether one or more gene expression products that correspond with the one or more genomic edits are involved with a pathological process of a pathological agent. The one or more genomic edits can comprise a modulation, a knockdown, or a knockout of an ACE2 gene and/or a modulation, a knockdown, or a knockout of one or more genes that are associated with polycystic kidney disease (PKD gene). Example PKD genes that can be modified include a polycystin-1 gene (PKD1), a polycystin-2 gene (PKD2) and/or a polycystic kidney and hepatic disease 1 gene (PKHD1). The PKD gene can be modulated in any manner suitable to contribute to a PKD phenotype, including genetic knock out or deletion. For example, one or both alleles of the PKD2 gene can be genetically knocked out to produce one or more PKD2 negative (e.g., PKD2^{-/-}) cells or cell types of the organoid. The resultant PKD model system can be used to characterize PKD disease pathogenesis and therapies for PKD or other conditions. In at least some instances, a gene “knock in” can be used as a genomic edit; an example allele that can be a knock in gene is a pathogenic form of apolipoprotein L-1 (APOL1).

Various methods can comprise an evaluation of a tropism or a response of one or more cells or cell types that comprise the human kidney organoid to a pathological agent. The tropism or the response can involve human kidney organoid proximal tubules, human kidney organoid distal tubules, polycystic kidney disease (PKD) cysts, and/or PKD cyst-lining epithelial cells. The response of the culture can comprise virus replication, cellular apoptosis, and/or disrupted cell morphology.

The maintenance medium can comprise an anti-pathological agent for at least potential interference with a pathological process. The pathological process can comprise infection with a virus, such as SARS-CoV-2, and/or replication of the virus, and the anti-pathological agent can comprise an antiviral agent; in the case of SARS-CoV-2, remdesivir, LCB1, FUS231-G10, and/or TRI2-2 may be utilized as the antiviral agent.

The disclosure also provides model systems and methods for use of viral vectors, such as SARS-CoV-2 viral vectors, for targeting the mature kidney epithelium, particularly the proximal tubules, among other structures of the kidney as well as other organs. The systems and methods can include and/or utilize gene therapy vectors for targeting the proximal tubules of the kidney.

Accordingly, the disclosure provides a method to virally transduce a human kidney cell, the method comprising: culturing a human kidney organoid in a human kidney organoid maintenance medium; contacting the culture with an agent that comprises a virus or a component of a virus; and characterizing a response of the culture to the agent; wherein the response of the culture informs development of viral transduction of the human kidney cell.

In at least some instances, characterizing the response of an organoid culture can comprise determining prevalence and efficiency of viral infection. The agent can comprise an adeno-associated virus (AAV) vector or a lentivirus vector, and in the case of an AAV vector, the AAV vector may comprise a serotype 2, a serotype 6, a serotype 8, or a serotype 9. If a lentivirus vector is used, the lentivirus vector can comprise, for example, a vesicular stomatitis virus envelope glycoprotein (VSV-G) pseudotype.

In at least some instances, the agent can comprise a SARS-CoV-2 virus vector. The SARS-CoV-2 virus vector can include one or more structural elements that are consistent with or derived from a SARS-CoV-2 virus. For targeted infection of a particular human kidney cell with the SARS-CoV-2 virus vector, the human kidney cell can be an element of a proximal tubule of the human kidney organoid, a distal tubule of the human kidney organoid, a polycystic kidney disease (PKD) cyst of the human kidney organoid, and/or a PKD cyst-lining epithelial cell of the human kidney organoid. Other human kidney cell types can be present in the organoid, such as podocytes.

The disclosure also provides methods for kidney-tropic gene delivery or gene therapy of a mature kidney epithelium of a human kidney, the method comprising contacting the mature kidney epithelium with a SARS-CoV-2 virus vector, a variant thereof, and/or a virus vector that comprises a component of an envelope and/or a spike protein of the SARS-CoV-2 virus vector or the variant. The mature kidney epithelium can comprise proximal tubules of the human kidney, however, other human kidney structures can be targeted, for example a distal tubule, a polycystic kidney disease (PKD) cyst, and/or a PKD cyst-lining epithelial cell of the human kidney. Other human kidney cell types, such as podocytes, can be present in the human kidney.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A shows a schematic of a kidney organoid infection protocol.

FIG. 1B shows whole well wide-field immunofluorescence images of iPS cell-derived organoids infected with SARS-CoV2-mNG. Arrows point to infected cells.

FIG. 1C shows a bar graph of results from qRT-PCR of SARS-CoV-2 envelope RNA in organoids infected with SARS-CoV-2 WA1 or mock-infected (MOCK). Mean±SEM of four independent experiments. Unpaired t-test, **p<0.01.

FIG. 1D shows a bar graph of results from plaque assays of SARS-CoV-2 infected human kidney organoids derived from iPS cells or ES cells. Mean±SEM of three and four independent experiments respectively. Unpaired t-test, ns p>0.05.

FIG. 1E shows representative confocal immunofluorescence images of organoids infected with SARS-CoV2-mNG.

FIG. 1F shows a bar graph of results from quantification of infected organoid cellular tropism. Mean±SEM of three experiments. 2-way ANOVA, multiple comparisons MOCK vs infected for each respective region. **p<0.01, ****p<0.0001, ns p>0.05.

FIG. 1G shows representative confocal immunofluorescence images of organoid infected with SARS-CoV2-GFP, with zoomed images of white boxed areas showing infected (top) versus uninfected (bottom) proximal tubules. Arrowheads indicate areas of disrupted LTL pattern. SARS-CoV2 efficiently infects human kidney organoids with tropism for proximal tubules.

FIG. 2A shows a schematic of a cystic PKD organoid infection protocol.

FIG. 2B shows representative confocal immunofluorescence images showing cystic PKD organoids infected with SARS-CoV2-mNG. Outlines denote independent organoids.

FIG. 2C shows a bar graph of results from quantification of infected organoid area (% total) of PKD and control (isogenic non-PKD) SARS-CoV-2-mNG infected cultures. Mean±SEM of three independent experiments each. Unpaired t-test, ns p>0.05.

FIG. 2D shows representative immunofluorescence images of cystic PKD organoids infected with SARS-CoV2-mNG, with zoom of cleaved caspase-3 staining and pyknotic nuclei.

FIG. 2E shows a bar graph of results from quantification of pyknotic nuclei and elevated cleaved caspase-3 levels of infected and non-infected cells of infected organoids. Mean±SEM of three independent experiments each. Unpaired t-test, **p<0.01, ***p<0.001. SARS-CoV-2 infects PKD organoid cystic epithelium.

FIG. 3A shows a line graph of prevalence of admission AKI, dialysis, and death in COVID⁺ patients over time.

FIG. 3B shows a table of viral variant mutations and characteristics.

FIG. 3C shows a bar graph of results from qRT-PCR of SARS-CoV-2 envelope RNA in infected kidney organoid cultures. Mean±SEM of three independent experiments. One-way ANOVA, Kruskal-Wallis post-hoc test, **p<0.01, ns p>0.05.

FIG. 3D shows a bar graph of results from plaque assays of SARS-CoV-2 infected kidney organoids. Mean±SEM of three independent experiments. One-way ANOVA, Kruskal-Wallis post-hoc test, *p<0.05, ns p>0.05. SARS-CoV-2 variants in kidney organoids reflect steady AKI prevalence in patients.

FIG. 4A shows a schematic of an ACE2 knockout and infection protocol.

FIG. 4B shows representative confocal immunofluorescence images of ACE2^{-/-} SARS-CoV2-mNG infected organoids, compared to isogenic controls.

FIG. 4C shows a bar graph of results from quantification of GFP⁺ area in ACE2^{-/-} organoids infected with SARS-CoV2-mNG, compared to mock-treated control. Mean±SEM of two experiments.

FIG. 4D shows a bar graph of results from a plaque assay of ACE2^{-/-} and control organoids infected with SARS-CoV-2 or mock-treated. Non-log scale is shown for this figure to emphasize low levels of infection in ACE2^{-/-} organoids. Mean±SEM of three independent experiments, utilizing two distinct mutant cell lines. One-way ANOVA with Tukey's post-hoc tests. *p<0.05, **p<0.01, ns p>0.05. ACE2 is the primary viral entry pathway for SARS-CoV-2 infection of kidney organoids.

FIG. 5A shows a schematic of a protocol for SARS-CoV-2 kidney organoid infection with remdesivir treatment.

FIG. 5B shows a graph of results from plaque assays of SARS-CoV2 and SARS-CoV-2-mNG infected kidney organoids treated with or without remdesivir. Mean±SEM of three independent experiments. Paired t-test, **p<0.01.

FIG. 5C shows a schematic of LCB1 binding to spike glycoprotein receptor binding domain (RBD).

FIG. 5D shows a schematic of LCB1 viral pre-treatment and infection of kidney organoids.

FIG. 5E shows a bar graph of results showing qRT-PCR expression levels of SARS-CoV-2 envelope RNA in infected kidney organoid cultures, with increasing levels of LCB1 protein pre-incubated with virus. Mean±SEM of four independent experiments, 2 iPS and 2 ES, normalized to beta-actin. One-way ANOVA, Kruskal-Wallis post-hoc test, *p<0.05, **p<0.01, ****p<0.0001, ns p>0.05.

FIG. 5F shows a bar graph of results from plaque assays of SARS-CoV-2 infected kidney organoids with increasing levels of LCB1 protein pre-incubated with virus. Mean±SEM or four independent experiments: 2 iPS and 2 ES. One-way ANOVA, Kruskal-Wallis post-hoc test, *p<0.05, **p<0.01, ****p<0.0001, ns>0.05. Therapeutic interventions reduce SARS-CoV-2 infection and replication in human kidney organoids.

FIG. 6 shows confocal immunofluorescent images of kidney organoids transduced with AAV-mCherry serotype 2, 8, 9.

FIG. 7 shows confocal immunofluorescent images of kidney organoids transduced with VSVG-lentivirus at different days of maturity.

FIG. 8 shows a bar graph of results from plaque assays of SARS-CoV-2 and SARS-CoV-2-mNG infected WT kidney organoids. Mean±SEM of four and three independent experiments respectively. Unpaired t-test, ns p>0.05.

FIG. 9A shows confocal immunofluorescent images of kidney organoids infected with SARS-CoV-2 pseudotyped lentivirus (Top panel) and Vero cells infected with SARS-CoV-2 pseudotyped lentivirus (Bottom panel).

FIG. 9B shows confocal immunofluorescent images of SARS-CoV-2 infected organoids.

FIG. 9C shows a bar graph of results from an LDH detection assay of infected kidney organoid supernatants 72 hours post SARS-CoV-2 infection. Mean±SEM of two independent experiments. Unpaired t-test, ns p>0.05.

FIG. 10A shows a bar graph of results from plaque assays of SARS-CoV-2 WT and SARS-CoV-2-mNG infected PKD human kidney organoids. Mean±SEM of three independent experiments. Unpaired t-test, ns p>0.05.

FIG. 10B shows confocal immunofluorescent images of SARS-CoV2-mNG infected cystic PKD organoids.

FIG. 11 shows a bar graph of results from an LDH assay of kidney organoid supernatant. Mean±SEM of three independent experiments. One-way ANOVA, Kruskal-Wallis post-hoc test, ns>0.05.

FIG. 12A shows light microscopy images of morphological changes that occur during culture of hPSC into tubular organoids over 21 days of culture.

FIG. 12B shows light microscopy images of non-PKD organoids (left) visibly compared to gene edited PKD organoids (right).

FIG. 12C shows confocal immunofluorescent images of Zika virus infected neural progenitors (left) and Herpes Simplex virus infected neural progenitors (right).

FIG. 13A shows a schematic of a workflow for a proteomic analysis of urine from patients who are either COVID-19 negative (BLUE) or COVID-19 positive (RED).

FIG. 13B shows box-and-whisker plots of results from proteomic analysis of urine proteins from patients who are either COVID-19 negative (BLUE (B)) or COVID-19 positive (RED (R)), compared with results from qPCR analysis of organoid mRNA from organoids that are either not infected with SARS-CoV-2 (BLUE) or infected with SARS-CoV-2 (RED).

FIG. 13C shows a scatter plot of results from proteomic analysis of urine proteins from patients who had lower levels of SARS-CoV-2 infection (left) compared to patients who had higher levels of SARS-CoV-2 infection (right).

FIG. 14 shows an overview of a workflow for making and using a human kidney organoid culture as a model system for characterizing a kidney pathology.

FIG. 15 shows a diagram of a system and method for making and using a human kidney organoid culture as a model system for characterizing a kidney pathology that may involve modifications to the organoid and/or the culture conditions.

FIG. 16A shows part of an image analysis workflow for a SARS-CoV-2-mNG infected WTC11 organoid that involves conversion of confocal Z-stacks to maximum intensity projections.

FIG. 16B shows part of an image analysis workflow for a SARS-CoV-2-mNG infected WTC11 organoid that involves split of multi-channel composite images into individual channels and the determination and application of uniform threshold parameters.

FIG. 16C shows part of an image analysis workflow for a SARS-CoV-2-mNG infected WTC11 organoid that involves use of Podocalyxin- and LTL-exclusive binary images as masks to generate images of SARS-CoV-2-mNG pixel intensity within each defined sub-region.

FIG. 16D shows part of an image analysis workflow for a SARS-CoV-2-mNG infected WTC11 organoid that involves determination of percent infection of each area for all organoids.

FIG. 17A shows a single-cell RNA sequencing (scRNAseq) reference plot from 13 COVID-19 positive patient urine samples.

FIG. 17B shows scRNAseq results of ISG15 expression by cell type.

FIG. 17C shows scRNAseq results of GALNT1 expression by cell type.

FIG. 18 shows results from an LDH assay of kidney organoid supernatant. Mean±SEM of three independent experiments. One-way ANOVA, Kruskal-Wallis post-hoc test, ns>0.05.

FIG. 19A shows immunofluorescent images of ACE2^{-/-} kidney organoid.

FIG. 19B shows immunofluorescent images of WTC11 kidney organoid.

FIG. 19C shows representative immunofluorescence images of ACE2 in cystic PKD organoids infected with SARS-CoV2-mNG.

FIG. 19D shows low- and high-magnification immunofluorescence images of cryosectioned kidney tissue (110 days). Images are representative of kidneys from five different sources.

FIG. 19E shows results from qRT-PCR analysis of ACE2 and TMPRSS2 expression in WTC11 mock and SARS-CoV-2 infected organoids. Mean±SEM of three independent experiments. Unpaired t-test, ns p>0.05.

FIG. 20A shows representative confocal immunofluorescence and phase images of remdesivir treated kidney organoids.

FIG. 20B shows representative confocal immunofluorescence images of live/dead stained PKD2^{-/-} and PKD2^{+/+} remdesivir treated organoids.

FIG. 20C shows fold change of SARS-CoV-2 replication of remdesivir treated organoids compared to DMSO treated controls.

FIG. 20D shows representative confocal immunofluorescence images of WTC11 SARS-CoV-2 infected organoids with and without 2 μM remdesivir treatment.

FIG. 21A shows results from a 24-hour APOL1 agonist screen.

FIG. 21B shows the effect of IFN-γ titration (10 ng/ml, 100 ng/ml, 1 mg/mL) on APOL1 levels.

FIG. 22A shows a table of example inhibitors, their targets, and their brief description.

FIG. 22B shows scRNAseq results for control+vehicle, control+1d IFN treatment, and IFN treatment in combination with JAK ½ inhibitors as shown for inhibition of APOL1 expression.

FIG. 22C shows scRNAseq results for control+vehicle, control+1d IFN treatment, control+3d IFN treatment, and IFN treatment in combination with JAK ½ inhibitors as shown for reversion of APOL1 expression.

FIG. 22D shows a pathway chart for canonical type-2 interferon signaling and APOL1 induction.

FIG. 23A shows results demonstrating that baricitinib prevents APOL1 upregulation in the organoid body.

FIG. 23B shows results demonstrating that baricitinib prevents APOL1 upregulation in EC vessels.

FIG. 23C shows results demonstrating that baricitinib prevents vascular regression with prolonged IFN.

FIG. 24A shows an example time course for prolonged IFN-γ treatment of organoids.

FIG. 24B shows no significant change in raw organoid size from prolonged IFN-γ treatment.

FIG. 24C shows that only tunicamycin treated organoids shrink.

FIG. 24D shows that IFN treatment does not result in enhanced loss of GFP-PODXL.

### DETAILED DESCRIPTION

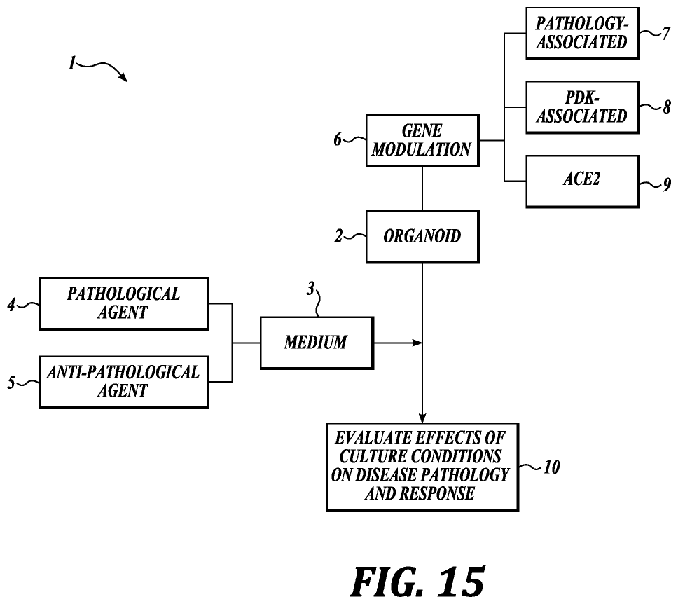

In general, the embodiments described herein provide phenotypic human kidney organoid model systems that comprise human kidney organoids which can be derived from induced pluripotent stem (iPS) cells and/or embryonic stem(ES) cells (FIG. 15). The organoids can be validated with clinical data and used as part of approaches for studying pathogenesis and implementing therapies for conditions, diseases, and disorders that impact the kidney, and gene therapy strategies that target the kidney or specific cells or cell types of the kidney.

The organoids can be comprised of cells with unedited genomes or, alternatively, may include one or more cells with one or more genomic edits, such as an insertion, a deletion, a frameshift, a replacement, a point mutation, a knock out, a knock in, or a different genomic edit. The use of genomic edits allows the determination and characterization of cellular factors and processes that contribute to or antagonize a pathology (e.g., SARS-CoV-2 pathology, acute kidney injury (AKI) pathology, chronic kidney disease (CKD) pathology, polycystic kidney disease (PKD) pathology, cystinosis (e.g., as can be modeled with knockout of a CTNS gene), and the like). In at least some aspects, a genomic edit allows cells, cell types, tissues, and/or organs that are otherwise not tractable for gene therapy with a viral vector to become tractable for gene therapy with the viral vector; for example, a genomic edit can cause increased expression of a factor that facilitates gene therapy with the viral vector and/or decreases resistance to gene therapy with the viral vector.

Further, the organoids can be comprised of one or more cells or cell types that are infected or capable of being infected with a virus, such as severe acute respiratory syndrome coronavirus 2 (SARS-CoV-2) virus. Infected organoids can be used to characterize pathological processes of the virus. These organoid model systems and methods also enable implementation of gene therapy strategies to target the kidney or specific cells or cell types of the kidney, such as the mature kidney epithelium, particularly the proximal tubules, in the case of SARS-CoV-2.

### DEFINITIONS

Unless specifically defined herein, all terms used herein have the same meaning as they would to one skilled in the art of the present invention. Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the terms “herein,” “above,” and “below,” and words of similar import, when used in this application, shall refer to this application as a whole and not to any particular portions of the application.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

“Viral preparation”, as used herein, refers to a composition, for example a solution, that includes a virus or a component of a virus. A viral preparation may be utilized in vitro or in vivo in combination with a cell, a cell type, an organoid, a tissue, an organ, an organism, or another biological system.

“SARS-CoV-2”, as used herein, refers to a severe acute respiratory syndrome coronavirus 2 virus or any variant thereof as disclosed herein, for example, a USA-WA1 (WA1) variant, a B.1.351-HV001 (Beta) variant, a B.1.1.7 (Alpha) variant, a P.1 (Gamma) variant, and/or a B.1.617.2 (Delta) variant (e.g., Isolate hCoV-19/USA/PHC658/2021 obtained from BEI Resources, Catalog No. NR-55611).

“Component”, as used herein, refers to a part or element of a larger whole. For example, a component of a SARS-CoV-2 spike protein may include a S1 subunit, a S2 subunit, a domain or motif of the S1 subunit, and/or a domain or motif of the S2 subunit.

“Infected”, as used herein, refers to a state of a biological system, such as a cell, a cell type, an organoid, a tissue, an organ, an organism, or another biological system, in which the biological system is invaded by or positive for a pathogen, for example, a virus.

“Differentially infected,” as used herein, refers to a state of a biological system in which there is increased invasion by or positivity for a pathogen compared to a reference level or, alternatively, a state of the biological system in which there is decreased or no invasion by or positivity for the pathogen compared to the reference level.

“Pathological agent”, as used herein, refers to a pathogen or infectious agent, or component thereof, that causes or contributes to a pathophysiology of a biological system, such as a cell, a cell type, an organoid, a tissue, an organ, an organism.

“Anti-pathological agent”, as used herein, refers to a molecule having at least one property or characteristic that is antagonistic to a process of a pathological agent or a component thereof.

“Antiviral agent”, as used herein, refers to an anti-pathological agent having at least one property or characteristic that is antagonistic to a process of a virus or a virus component. Example antiviral agents include a small molecule, a nucleotide analog prodrug (e.g., remdesivir), a nucleic acid (e.g., DNA, RNA), a protein, and a miniprotein inhibitor.

“Miniprotein inhibitor”, as used herein, refers to any of various short polypeptides (e.g., about 56-amino acid residues in length) that bind the SARS-CoV-2 receptor-binding domain (RBD) with high affinity and potently neutralize authentic virus in cell culture with half-maximal effective concentration (EC50) values in the picomolar range (e.g., EC₅₀<30 pM). Example miniprotein inhibitors include LCB1 (also referred to as MON1), FUS231-G10, and TRI2-2 (a homotrimeric version of the 75-residue ACE2 mimic AHB2).

“Gene knockout”, and “genetically knocking out”, as used herein, refer to a procedure to mutate DNA of a gene in a manner that inhibits expression of the gene permanently. An example knockout procedure can implement CRISPR genome editing.

“Tropism”, as used herein, refers to the change of all or part of a biological system in a particular manner in response to an external stimulus. An example of tropism is a biological change of all or part of a human kidney organoid in response to contact with a viral preparation or a pathological agent. Example biological systems include a cell, a cell type, an organoid, a tissue, an organ, and an organism.

“Transduction”, as used herein, refers to a virus-mediated introduction of genetic material into a biological system such as a cell, a cell type, an organoid, a tissue, an organ, an organism, or another biological system.

### HUMAN KIDNEY ORGANOID SYSTEMS AND METHODS

Generally, a human kidney organoid model system is provided for characterization of a kidney pathophysiology. A method for characterizing a SARS-CoV-2 infection includes generating a culture that comprises the human kidney organoid and a human kidney organoid maintenance medium for the culturing of the human kidney organoid, contacting the culture with a viral preparation that comprises a severe acute respiratory syndrome coronavirus 2 (SARS-CoV-2) virus or a variant or component thereof, and determining whether one or more cells or cell types of the human kidney organoid are infected by the viral preparation and produce a SARS-CoV-2 infected human kidney organoid. The method can involve modeling SARS-CoV-2 infection of the kidney, determining the cells or cell types targeted by the virus, and/or evaluating compositions and methods for treatment or prevention of kidney pathologies associated with SARS-CoV-2 infection or COVID-19.

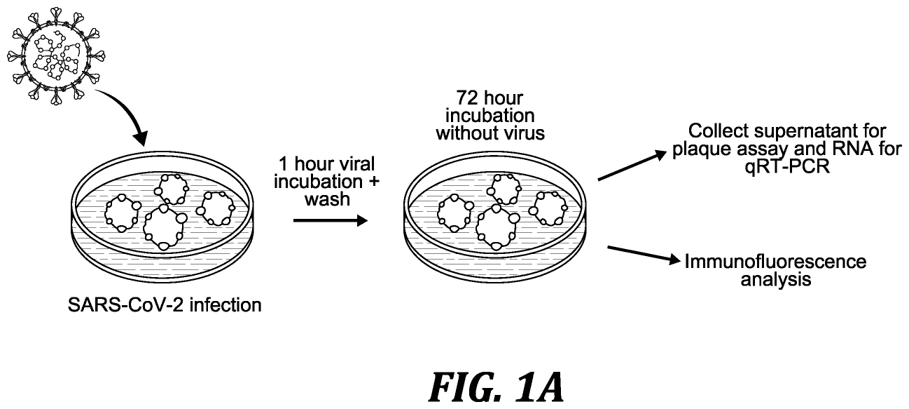

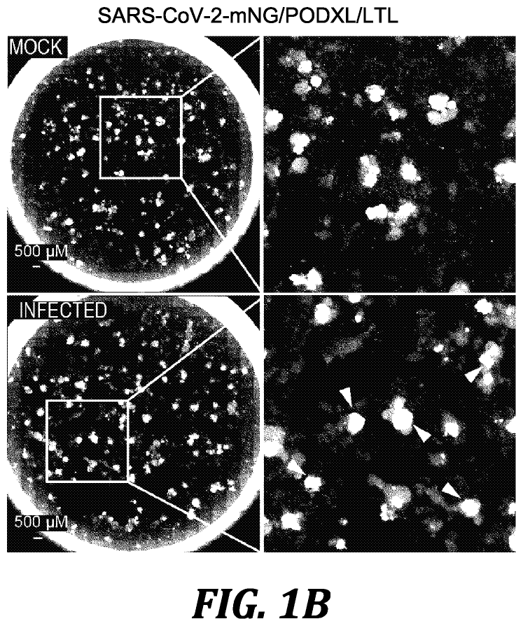

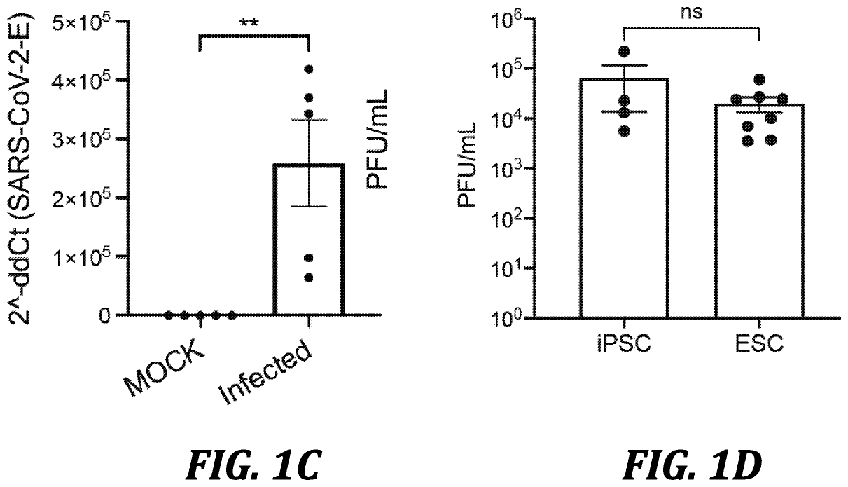

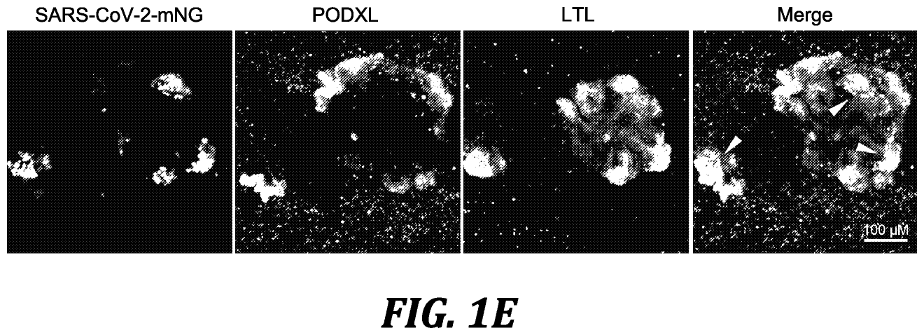

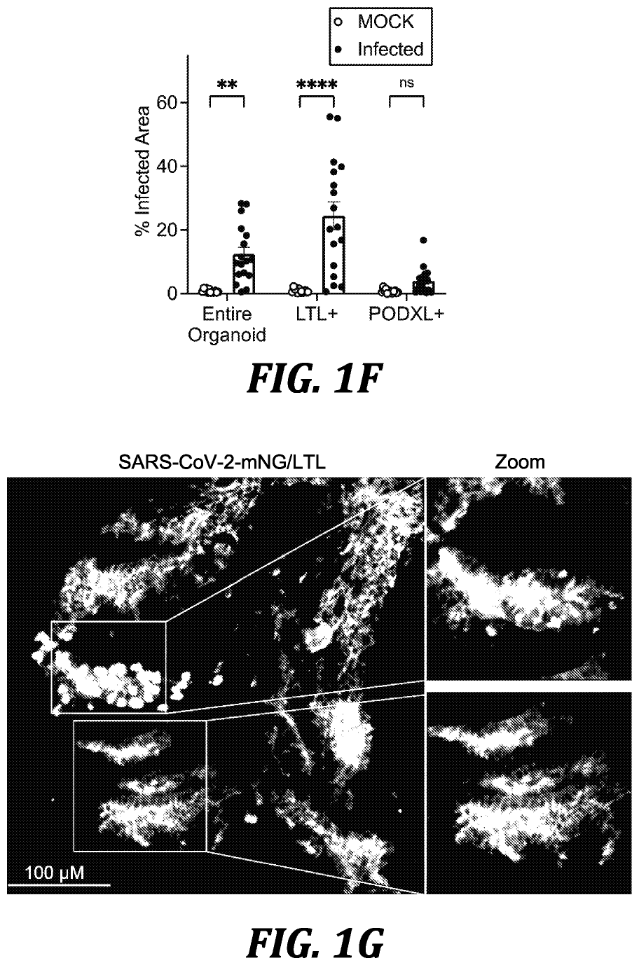

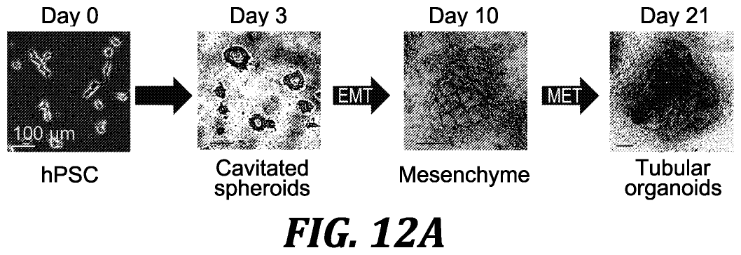

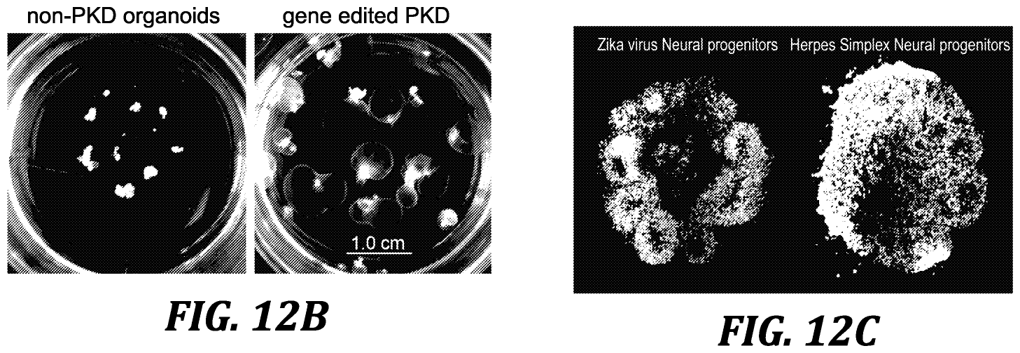

The human kidney organoid culture can be generated as a result of a procedure for differentiating a human iPS cell culture and/or a human ES cell culture into organoids, for example, an existing human kidney organoid differentiation procedure (FIG. 12A). The maintenance medium can be comprised of a set of ingredients or factors for growing, culturing, and/or maintaining the organoid in the culture. The viral preparation can be comprised of the SARS-CoV-2 virus or variant or component thereof in combination with any suitable salts, additives, carriers, or excipients. The one or more cells or cell types of the organoid can be determined as being infected (or not infected) with the virus by plaque assay, qRT-PCR, immunofluorescence analysis, and/or a different analysis (see, e.g., FIG. 1A).

In embodiments, one or more cells or cell types of the human kidney organoid are angiotensin converting enzyme 2 (ACE2) positive. One or more ACE2 gene alleles can be present, intact, and expressed in all or a subset of cells or cell types of the organoid, and as described elsewhere herein with examples, this enables SARS-CoV-2 virus to infect those ACE2 positive cells or cell types. Since at least some cells or cell types of the organoid can not be ACE2 positive (or may not be sufficiently ACE2 positive), they may not ordinarily be infected with SARS-CoV-2 virus. As a result, in at least some embodiments, a subset of cells or cell types of the organoid (e.g., the mature kidney epithelium, particularly the proximal tubules as disclosed herein) are ACE2 positive and are infectable with SARS-CoV-2 virus.

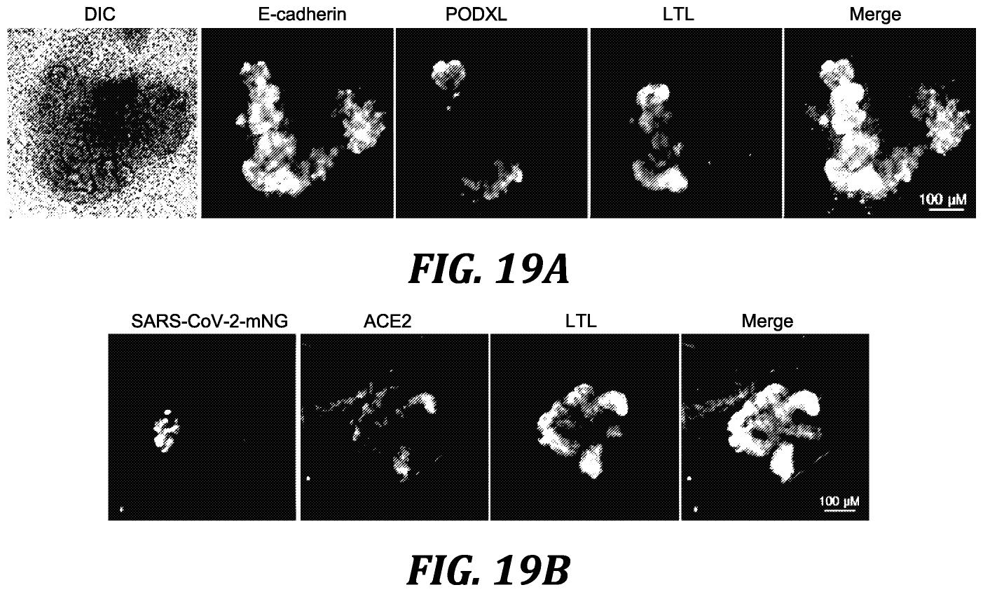

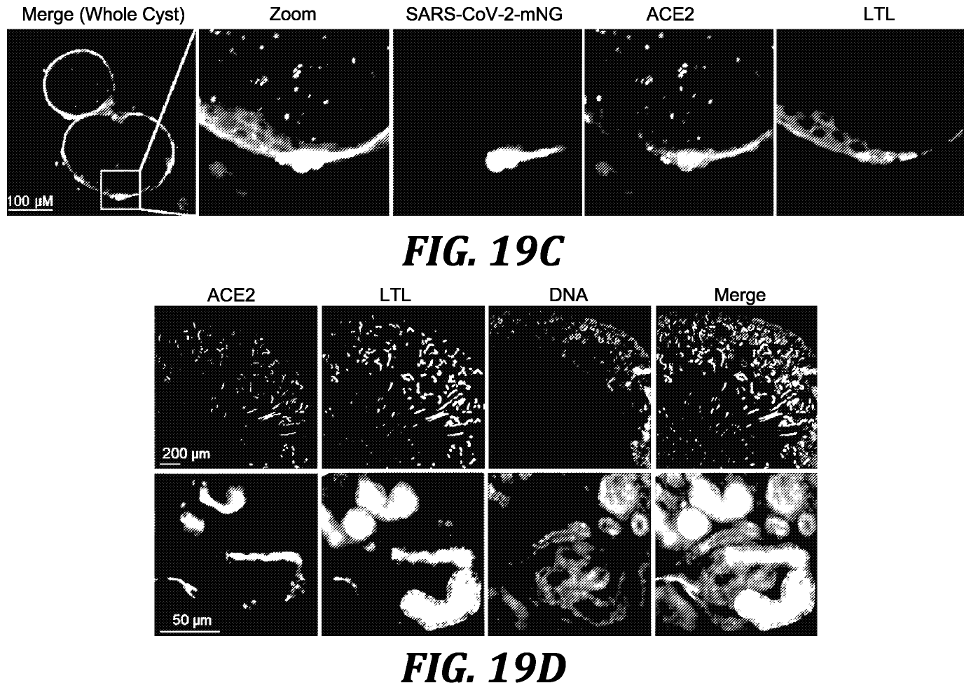

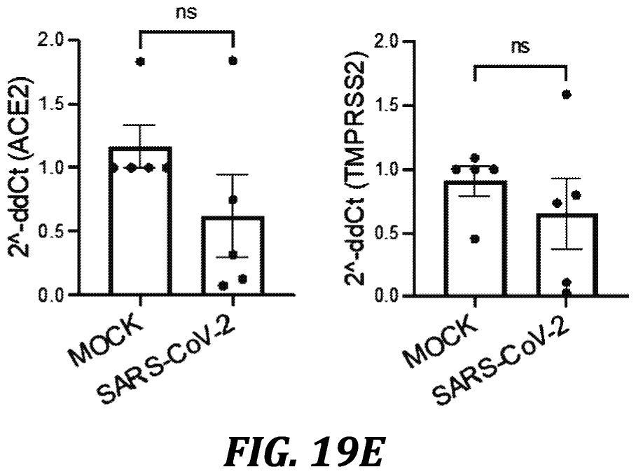

Since ACE2 contributes to infectability of kidney cells, various methods can involve modulating an ACE2 gene in one or more cells or cell types of the human kidney organoid to produce one or more ACE2 modulated cells or cell types. After the culture is contacted with the viral preparation, it may be determined whether the one or more ACE2 modulated cells or cell types are differentially infected by the viral preparation relative to one or more cells or cell types that are not ACE2 modulated (e.g., one or more cells or cell types that do not have modulated ACE2). For example, ACE2 can be knocked down or, alternatively, knocked out, in one or more cells or cell types of the human kidney organoid to produce one or more ACE2 negative cells or cell types, and after contacting the culture with the viral preparation, it can be determined whether the one or more ACE2 negative cells or cell types are not infected by the viral preparation (FIG. 19A). As another example, ACE2 expression can be wild-type (FIG. 19B) or increased (e.g., with genetic engineering or introduction of a transgene for expression by one or more cells or cell types of the organoid) to increase susceptibility of one or more cells or cell types of the organoid to SARS-CoV-2 infection.

The organoid model system can be representative of, or can correspond with, a particular kidney condition, disease, or disorder. For example, one or more cells or cell types of the human kidney organoid can have a genetic modification that is associated with polycystic kidney disease (e.g., a “PKD genotype”) (see FIG. 12B and FIG. 12C). The PKD genotype can be created to produce one or more PKD genotype cells or cell types to be evaluated, after contacting the culture with the viral preparation, for whether the one or more PKD genotype cells or cell types are infected or differentially infected by the viral preparation. In instances where cysts of the PKD genotype organoids are infected or differentially infected, this can inform development of gene therapy techniques for targeting PKD cysts with gene therapy.

While any suitable PKD gene or genes can be modulated to produce the PKD genotype (e.g., a polycystin-1 gene (PKD1), a polycystin-2 gene (PKD2) and/or a polycystic kidney and hepatic disease 1 gene (PKHD1)), in certain embodiments, the PKD genotype includes a PKD2 gene knockout in one or more cells or cell types of the human kidney organoid to produce one or more PKD2 negative cells or cell types. The method includes determining, after contacting the culture with the viral preparation, whether the one or more PKD2 negative cells or cell types are infected or differentially infected by the viral preparation. In at least some embodiments, a gene “knock in” can be used as a genomic edit; an example allele that may be a knock in gene is a pathogenic form of APOL1.

The tropism or response of the organoid to a pathological agent, such as a SARS-CoV-2 virus or variant or component thereof, can be evaluated. The tropism or response can involve the organoid as a whole or, alternatively, can only involve certain cells or cell types of the organoid, such as human kidney organoid proximal tubules, human kidney organoid distal tubules, polycystic kidney disease (PKD) cysts, and/or PKD cyst-lining epithelial cells.

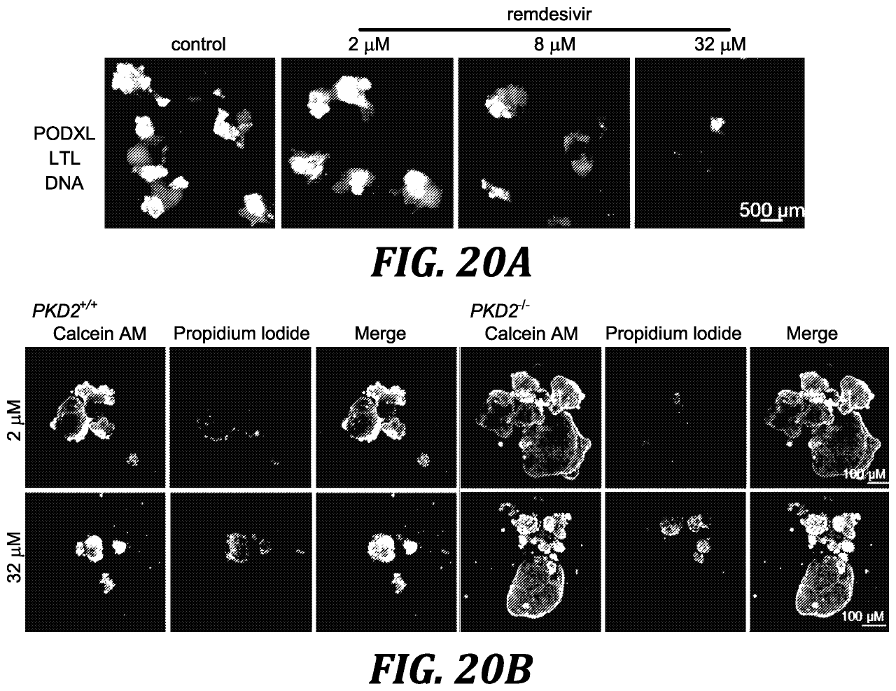

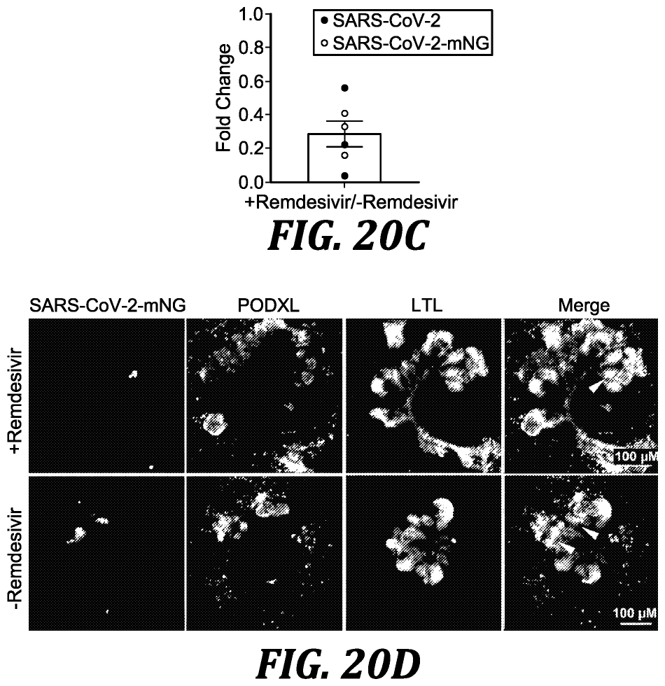

The human kidney organoid culture can be a model system for a kidney disease pathology including for evaluating therapeutic or at least potentially therapeutic agents for their ability to interfere with, impede, inhibit, and/or antagonize a pathological process that involves a kidney. As such, the maintenance medium can include an anti- pathological agent, such as an anti-viral agent, for at least potential interference with a pathological process, such as infection with a virus and/or replication of the virus. In example embodiments, the antiviral agent can comprise the nucleotide analogue prodrug remdesivir and/or a miniprotein inhibitor (e.g., LCB1, FUS231-510, and/or TRI2-2), however, other antiviral agents may be utilized without departing from the scope of the disclosure (see FIG. 20A, FIG. 20B, FIG. 20C, and FIG. 20D).

Human kidney cell exposure to SARS-CoV-2 viruses, including variants and/or components thereof, is associated with elevated IFN-γ and elevated APOL1, which causes nephropathy. As such, the human kidney organoid model system can be used for treatments for inhibiting and/or reverting APOL1 expression for prevention of nephropathy, for example, as part of a treatment for COVID-19, for example, for patients that have a kidney condition and can be more susceptible to serious COVID-19.

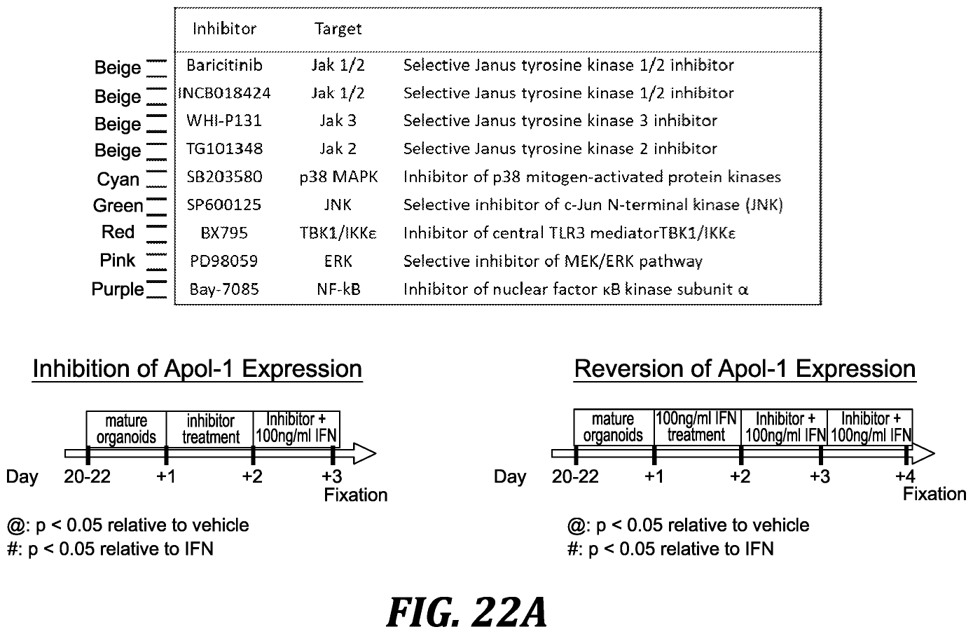

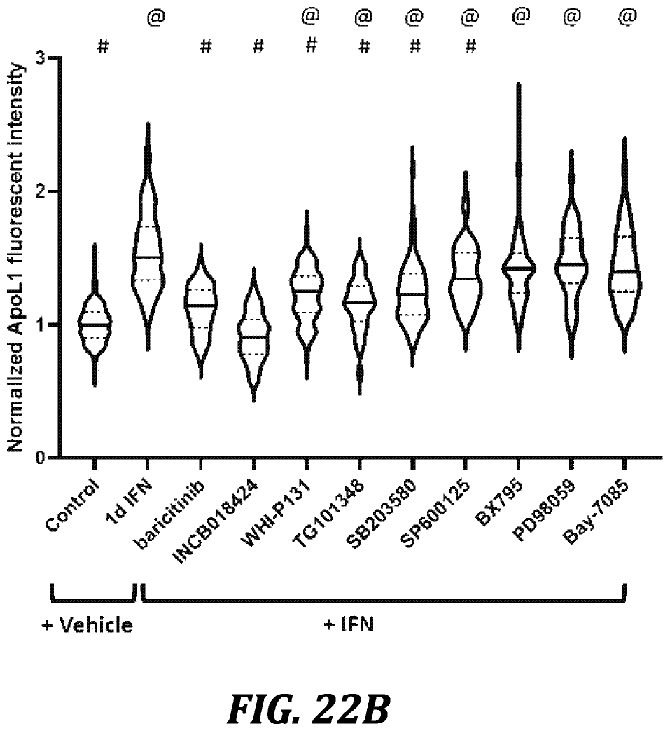

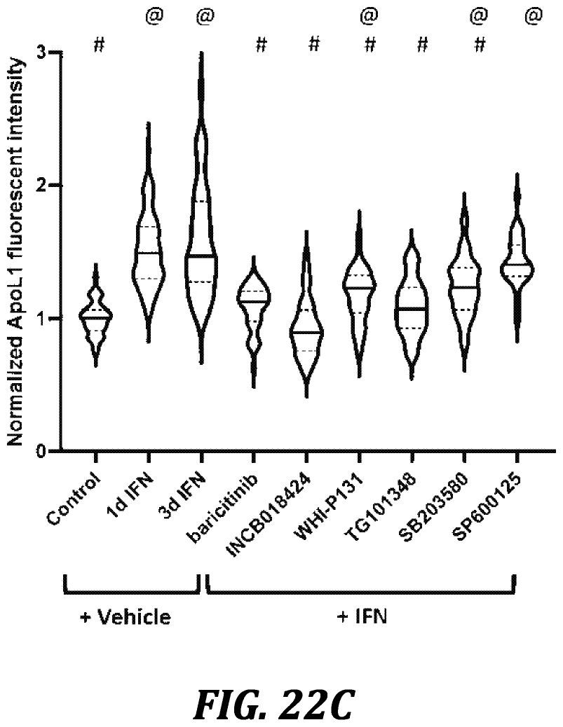

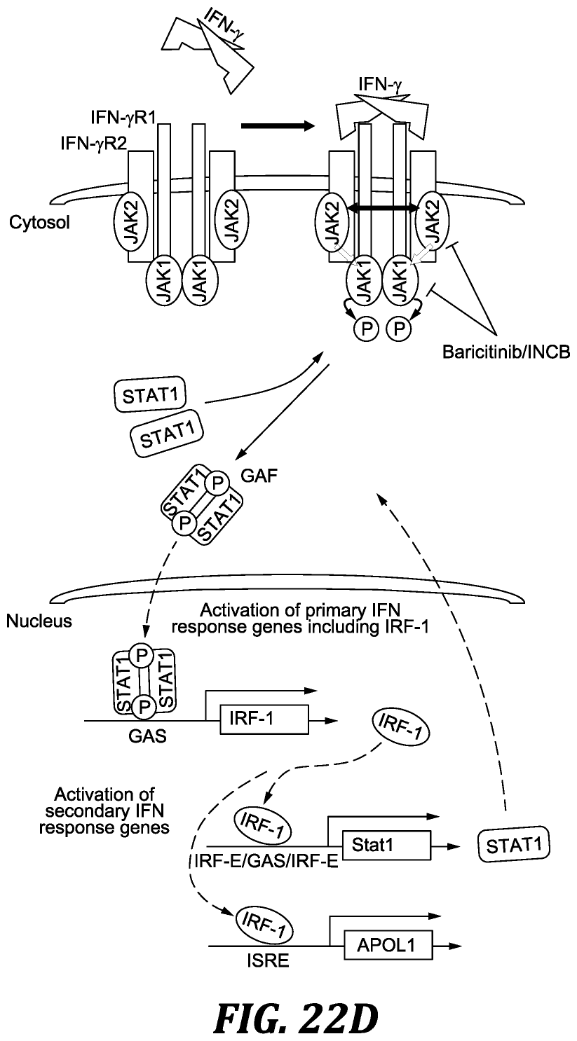

Accordingly, the culture can be contacted with a molecule for at least partial inhibition and/or reversion of APOL1 expression. The molecule can be selected from any suitable group of inhibitors for combating nephropathy, however, in certain embodiments, the inhibitor can comprise one or more inhibitors selected from a group that includes: baricitinib, INCB018424, WHI-P131, TG101348, SB203580, SP600125, BX795, PD98059, Bay-7085, and any combination thereof (see, for example, FIG. 22A). In certain embodiments, the molecule comprises one or more JAK ½ inhibitors, such as baricitinib and/or INCB018424.

In instances where one or more cells or cell types of the organoid are infected with a SARS-CoV-2 virus, a resultant SARS-CoV-2 infected organoid model system can be produced and used subsequently in methods for characterizing a pathophysiology. These methods involve culturing a culture that comprises a SARS-CoV-2 infected human kidney organoid and a human kidney organoid maintenance medium for maintenance of the human kidney organoid, contacting the culture with a pathological agent, and characterizing a response of the culture to the pathological agent. The pathological agent can be a virus or a non-virus pathological agent, such as a toxin or a bacterium. The response of the culture can involve a tropism or response of one or more cells or cell types of the organoid, which can involve, among other possible cells or cell types, human kidney organoid proximal tubules, human kidney organoid distal tubules, polycystic kidney disease (PKD) cysts, and/or PKD cyst-lining epithelial cells. The pathological agent can comprise a SARS-CoV-2 virus or a variant or a component of a SARS-CoV-2 virus, and the response of the culture may comprise SARS-CoV-2 virus replication, cellular apoptosis, and/or disrupted cell morphology, for example, as evidenced by immunofluorescence microscopy.

The characterization of the kidney pathophysiology can include determination of whether one or more gene expression products that correspond with the one or more genomic edits are involved with a pathological process. The one or more genomic edits can comprise a modulation, a knockdown, or a knockout of an ACE2 gene, and/or a modulation, a knockdown, or a knockout of one or more genes that are associated with polycystic kidney disease (PKD gene). The PKD gene may comprise a polycystin-1 gene (PKD1), a polycystin-2 gene (PKD2) and/or a polycystic kidney and hepatic disease 1 gene (PKHD1). In these and other embodiments the method can further comprise contacting the culture with an anti-pathological agent for at least potential interference with a pathological process of the pathological agent. In such embodiments, the pathological process can comprise infection with a SARS-CoV-2 virus and/or replication of the SARS-CoV-2 virus and the anti-pathological agent can comprise an antiviral agent. The antiviral agent can comprise, for example, the nucleotide analogue prodrug remdesivir and/or a miniprotein inhibitor (e.g., LCB1, FUS231-510, and/or TRI2-2). In at least some embodiments, a gene “knock in” can be used as a genomic edit; an example allele that can be a knock in gene is a pathogenic form of APOL1.

In at least some instances, signaling of the IFN-γ pathway can be elevated because of exposure of a human kidney organoid or a human kidney to a SARS-CoV-2 virus or variant or viral component, and as a result, APOL1 can be elevated which can lead to nephropathy. As such, the pathophysiology, which may involve nephropathy, can be further characterized, or managed, by contacting a kidney cell with a molecule for at least partial inhibition and/or reversion of APOL1 expression and nephropathy. The molecule can be selected from any suitable group of inhibitors for combating nephropathy, however, in certain embodiments, the inhibitor can comprise one or more inhibitors selected from a group that includes: baricitinib, INCB018424, WHI-P131, TG101348, SB203580, SP600125, BX795, PD98059, Bay-7085, and any combination thereof (see, for example, FIG. 22A). In certain embodiments, the molecule is one or more JAK ½ inhibitors, such as baricitinib and/or INCB018424.

### HUMAN KIDNEY CELL TRANSDUCTION METHODS

In general, a method to virally transduce a human kidney cell involves culturing a human kidney organoid in a human kidney organoid maintenance medium, contacting the culture with an agent that comprises a virus or a component of a virus, and characterizing a response of the culture to the agent. The response of the culture, e.g., a response that indicates which cells or cell types of the organoid are infected, to what level they are infected, and/or how they respond to infection (e.g., prevalence and efficiency of infection), helps inform development of viral transduction of the human kidney cell. Improved methods for viral transduction of human kidney cells can be implemented in vitro using an organoid model system as disclosed herein, or alternatively, can be implemented in vivo using a test subject. As such, the human kidney organoid model system can inform development of gene therapy strategies for use in human kidneys in vivo.

The agent can comprise an adeno-associated virus (AAV) vector or a lentivirus vector, for example, an AAV vector that comprises a serotype 2, a serotype 6, a serotype 8, or a serotype 9, and/or a lentivirus vector that comprises a vesicular stomatitis virus envelope glycoprotein (VSV-G) pseudotype. However, as described herein, these virus vectors (i.e., AAV, VSV-G lentivirus) are ineffective at targeting kidney organoids, and as such, a different virus vector can be used, such as a SARS-CoV-2 virus vector, which as described herein can target a proximal tubule of the human kidney organoid, a distal tubule of the human kidney organoid, a polycystic kidney disease (PKD) cyst of the human kidney organoid, and/or a PKD cyst-lining epithelial cell of the human kidney organoid.

As a result, a method for kidney-tropic gene delivery or gene therapy of a mature kidney epithelium of a human kidney is provided. The method includes contacting the mature kidney epithelium with a SARS-CoV-2 virus vector, a variant thereof, and/or a virus vector that includes a feature, e.g., an envelope and/or a spike protein, of the SARS-CoV-2 virus vector. Since SARS-CoV-2 virus targets the mature kidney epithelium, the SARS-CoV-2 virus vector can be used to target the mature kidney epithelium, for example, the proximal tubules of the human kidney, for gene therapy. However, other kidney structures can be targeted by the SARS-CoV-2 virus vector, such as a distal tubule, a polycystic kidney disease (PKD) cyst, and/or a PKD cyst-lining epithelial cell of the human kidney. An example of an application of a SARS-CoV-2 virus vector gene therapy includes delivery of a CTNS gene into proximal tubules of a patient with cystinosis.

### EXAMPLES

The following are examples of human kidney organoid model systems and methods for making and using human kidney organoid model systems. These examples are meant to enable a person to practice the invention and are not intended to be limiting to the disclosure or any claims that may refer to one or more of these examples or features thereof.

### Example 1: SARS-CoV-2 Infects Organoid Proximal Tubules with Pathogenic Effects

Kidneys are critical target organs of COVID-19, but susceptibility to and responses from infection remain poorly understood. In this example, SARS-CoV-2 variants are combined with genome edited kidney organoids and clinical data to investigate tropism, mechanism, and therapeutics. It is shown that SARS-CoV-2 specifically infects organoid proximal tubules amongst diverse cell types. Infections produce replicating virus, apoptosis, and disrupted cell morphology, including in the context of polycystic kidney disease (PKD). Infection is ameliorated in ACE2^{-/-} organoids and blocked via treatment with antiviral agents. Collectively, these studies clarify the impact of kidney infection in COVID-19 as reflected in organoids and clinical populations, enabling assessment of viral fitness and emerging therapies. Additionally, both VSVG pseudotyped lentiviruses and AAVs 2, 6, 8, and 9 were unable to transduce mature organoids at high levels, indicating that SARS-CoV-2 is an advantageous delivery strategy for targeting the mature kidney epithelium.

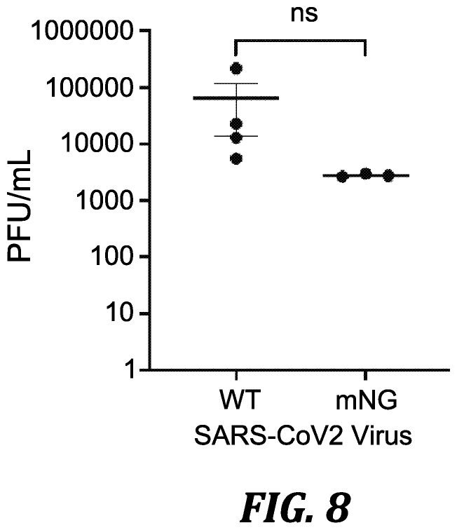

To assess the susceptibility of kidney organoid cell types to SARS-CoV-2 infection, human kidney organoids were exposed to a multiplicity of infection (MOI) 10 of SARS-CoV-2/WA1 (SARS49 CoV-2) and infection was measured 72 hours later (FIG. 1A). Using SARS-CoV-2 genetically engineered to express mNeonGreen (SARS-CoV-2-mNG), it was observed that the fluorescent signal localized in specific kidney organoid structures (FIG. 1B; see also FIG. 8). Viral RNA of SARS-CoV-2 was readily detected in infected cultures, indicating that virus had entered cells (FIG. 1C). 72 hours post-infection, supernatants from organoids exposed to SARS-CoV-2 or SARS-CoV-2-mNG efficiently infected Vero cells and produced viral plaques, demonstrating functional virion production in kidney organoids (FIG. 1D). Immunofluorescence analysis of nephron markers in organoids exposed to SARS-CoV-2-mNG revealed specific infection of Lotus tetragonolobus lectin positive (LTL⁺) proximal tubules (FIG. 1E). Using semi-automated image analysis quantification, it was found that 12.4% of the total organoid area and 24.5% of the total LTL⁺ area was infected, respectively, whereas infection of podocytes (PODXL⁺) was not significant (FIG. 1F). Close inspection of infected versus non-infected proximal tubules revealed swollen, rounded cells with a disruption of smooth LTL patterning at the apical plasma membrane (FIG. 1G).

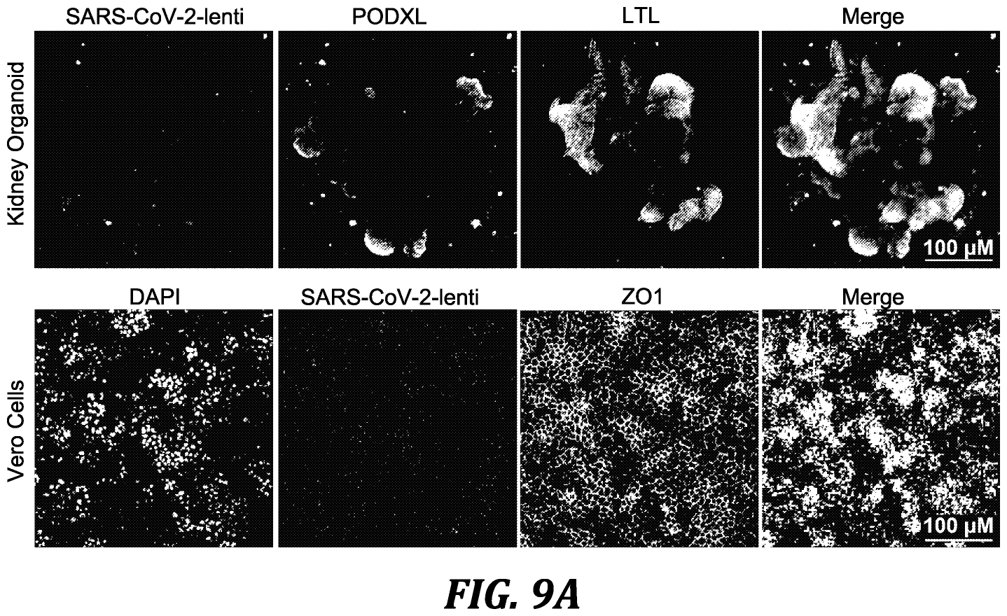

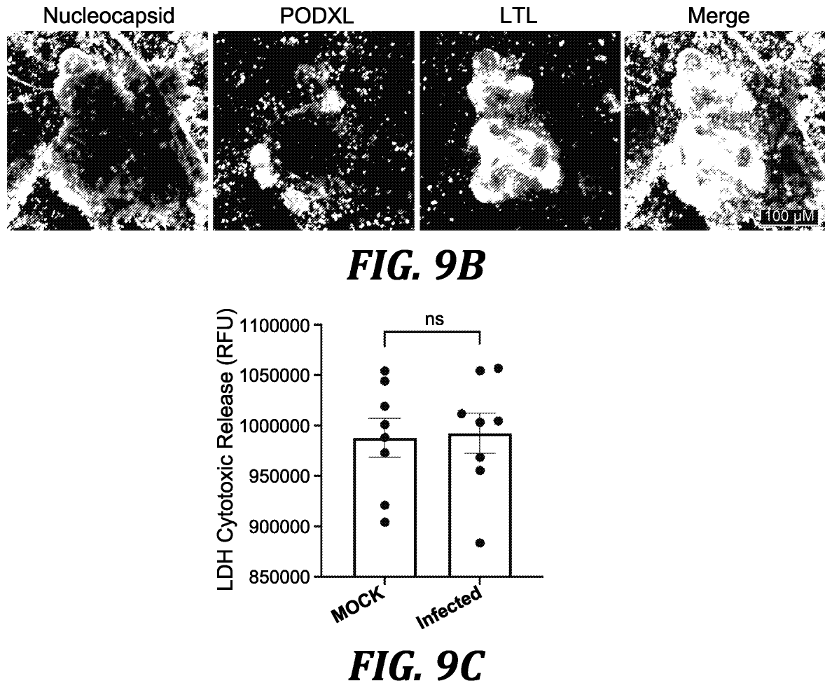

It was found that the use of SARS-CoV-2-mNG was vital for establishing the tropism of infection in kidney organoids. In contrast to SARS-CoV-2-mNG, a commercially available green fluorescent protein (GFP) expressing lentivirus pseudotyped for SARS-CoV-2 failed to productively infect kidney organoids or Vero cells (FIG. 9A). This likely reflects inferior levels of infection by SARS pseudotyped lentiviruses, compared to native virus. In addition, a commercially available antibody raised against SARS-CoV-2 nucleocapsid did not produce specific staining in organoids infected with SARS-CoV-2, but rather showed high background staining levels in stromal cells (FIG. 9B). Both the pseudotyped lentivirus and the antibody are commercial products that were generated shortly after the COVID-19 pandemic started. It was concluded that these products were inferior to actual coronavirus, the use of which was necessary to accurately monitor infection by SARS-CoV-2 in the organoids. Lactate dehydrogenase (LDH) release was not detectably increased in infected organoid supernatants, consistent with the observation that SARS-CoV-2 treatment was not overtly toxic to these cultures as a whole (FIG. 9C). These findings reveal specific proximal tubular tropism of SARS-CoV-2 capable of producing replicating virus and disrupting cell morphology, as assessed by direct measurements and state-of-the-art tools.

### SARS-CoV-2 Infects PKD Cystic Epithelium Causing Cytotoxicity

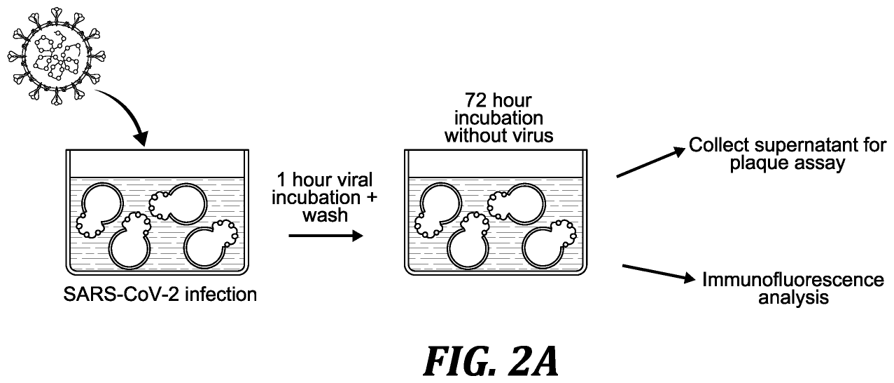

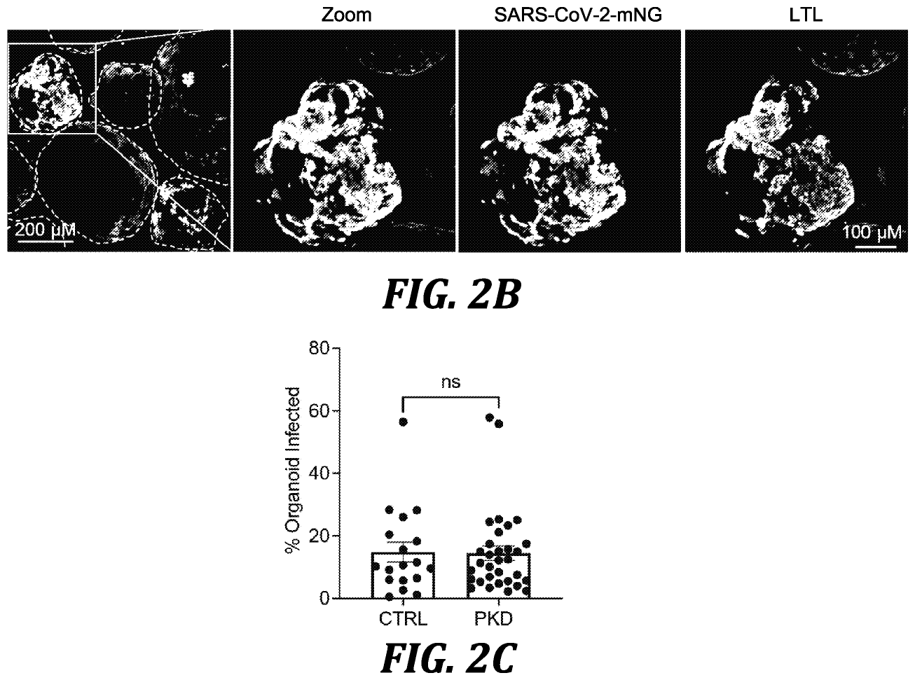

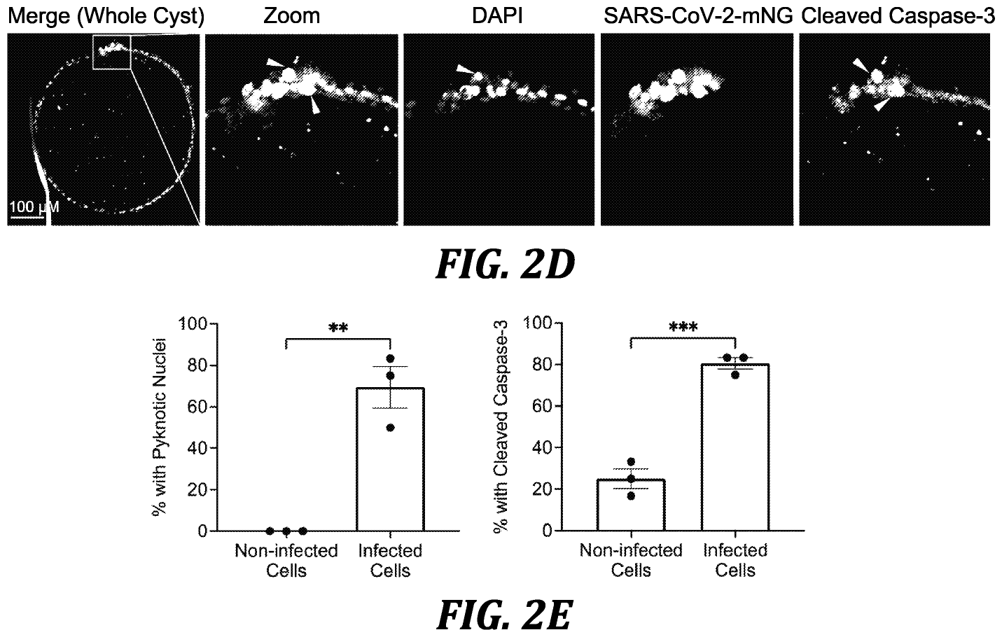

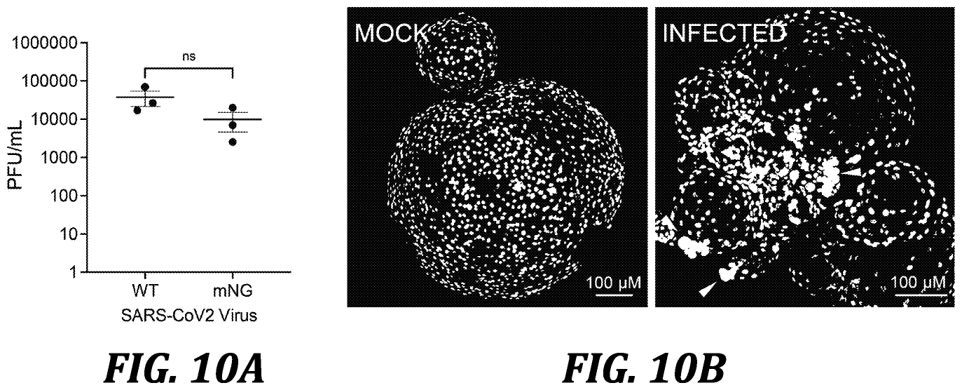

It was assessed whether PKD cysts are susceptible to SARS-CoV-2 infection with PKD2^{-/-} organoids in suspension culture, which form cysts from proximal and distal tubules (FIG. 2A). Cystic organoids were infected with SARS-CoV-2 and SARS-CoV-2-mNG and assessed for viral infection and replication via plaque assay and immunofluorescence staining (FIG. 2B and FIG. 10A). A subpopulation of cyst-lining epithelial cells with LTL binding affinity (suggesting a proximal tubular origin) was infected selectively by SARS-CoV-2-mNG and caused cell swelling (FIG. 2B and FIG. 10B; see also FIG. 19C and FIG. 19D). The percentage of infected area per cystic organoid was comparable to that of non-cystic organoids (FIG. 2C). Infection-induced apoptosis of cystic PKD epithelium was observed in infected organoids as indicated by significantly increased expression of cleaved caspase-3 and pyknotic nuclei in infected cells compared to non-infected cells in the same organoid (FIG. 2D and FIG. 2E). Together, this data indicates that PKD cysts derived from proximal tubules express ACE2 and are susceptible to SARS-CoV-2 infection, and that infection induces apoptosis in cystic epithelium.

### SARS-CoV-2 Variants in Kidney Organoids Reflect Steady AKI Prevalence in Patients

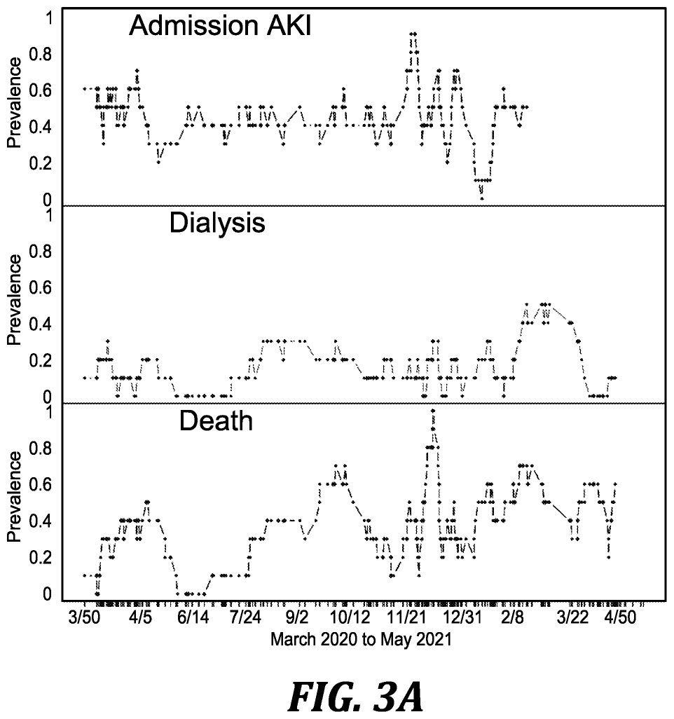

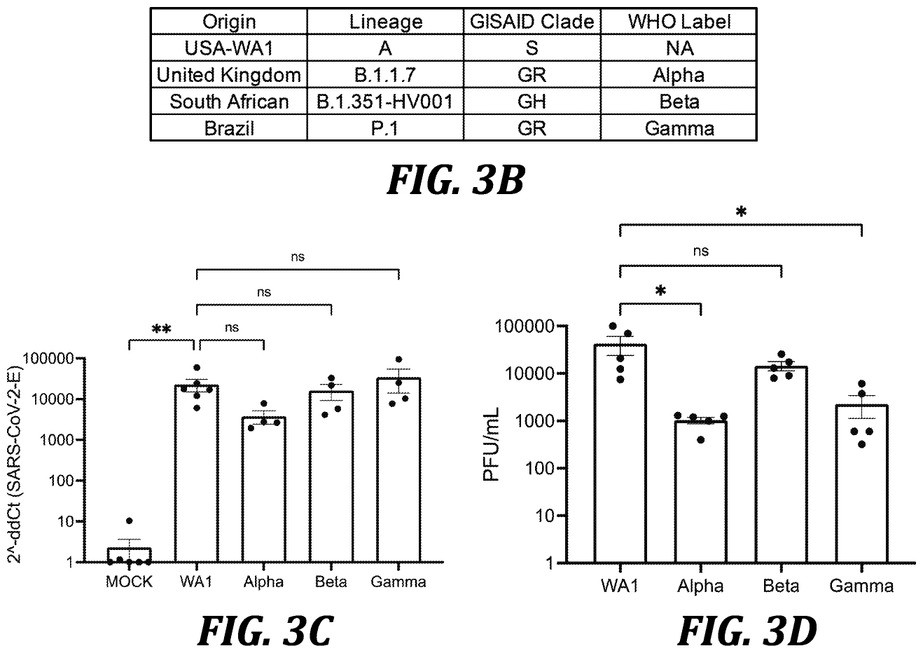

It is unknown if SARS-CoV-2 variants of concern (VOC) have different tropism to extra-pulmonary organs, such as the kidney. To assess whether rates of admission AKI, dialysis, or death change over time, the prevalence at each patient's admission, of admission AKI, inpatient dialysis, and in-hospital death were plotted over time using that patient and the next nine COVID-19⁺ patients admitted to the ICU (FIG. 3A). Death, dialysis, and AKI all had relatively low variance over time: AKI hovering around 40% prevalence, dialysis around 20%, and death around 50% between March 2020 to February 2021. While comprehensive VOC sequencing data was unavailable in this COVID-19⁺ patient cohort, in Washington State, US, the WA1 variant was the predominant viral strain in March 2021, and the Alpha, Beta, and Gamma viral variants of concern were detected in the US in January 2021, while rates of AKI and dialysis remained steady.

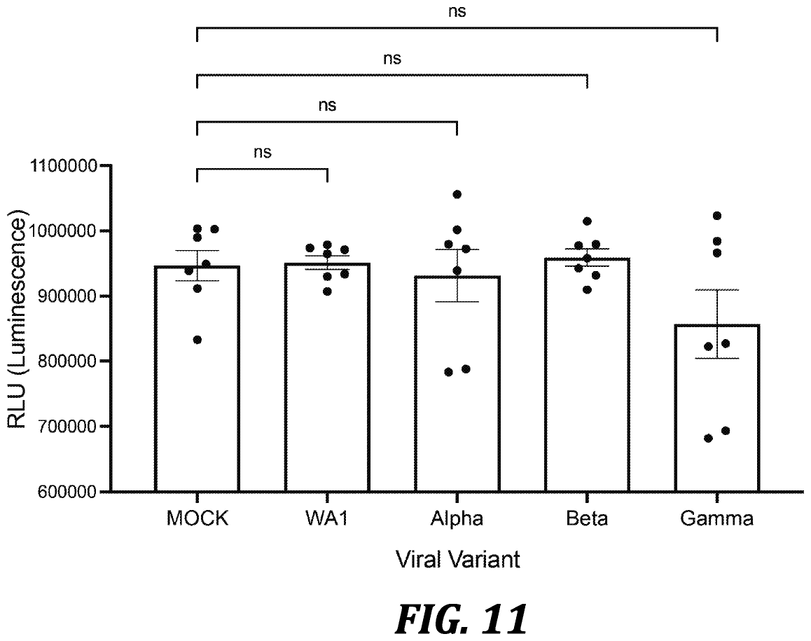

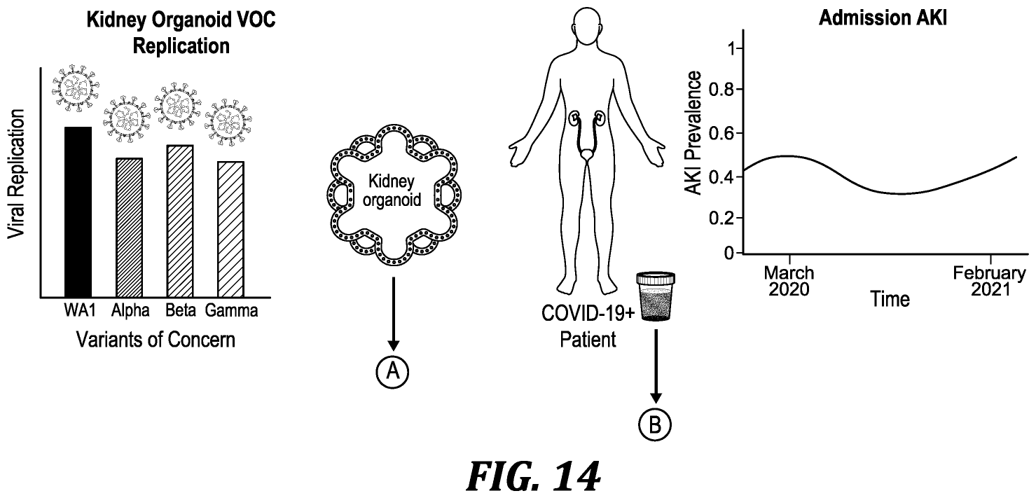

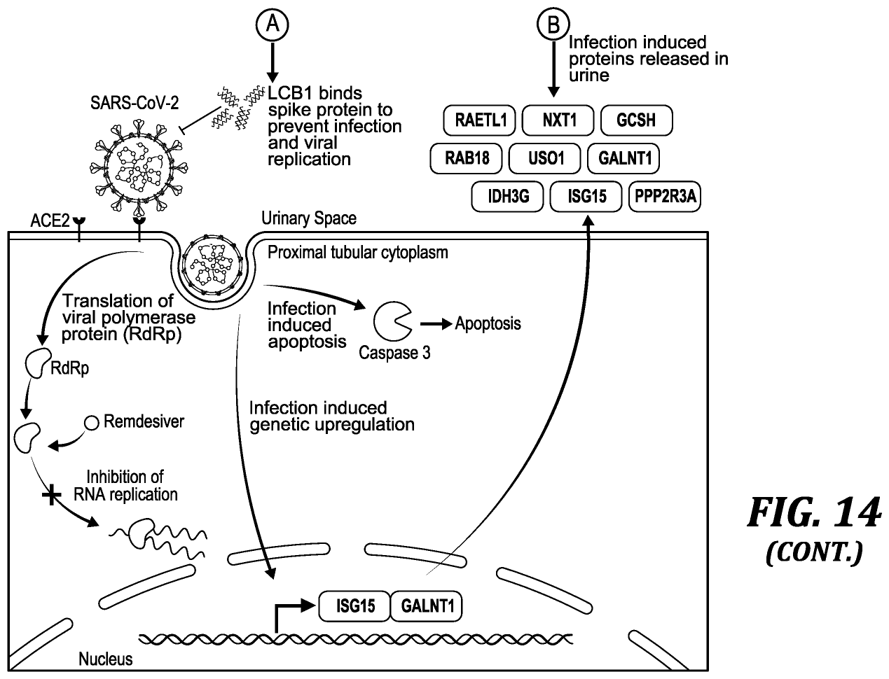

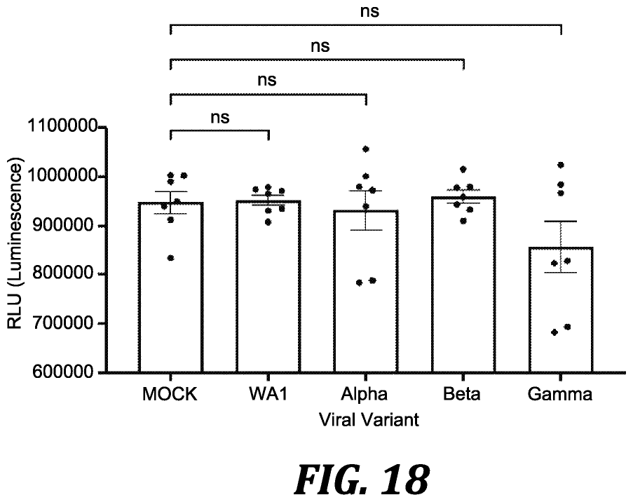

To assess whether emerging SARS-CoV-2 VOC exhibit altered viral fitness in kidney organoids, kidney organoids were infected with four viral variants: USA-WA1 (WA1), B.1.351-HV001 (Beta), B.1.1.7 (Alpha), and P.1 (Gamma) (FIG. 3B). qRT-PCR analysis of RNA extracted from infected organoids demonstrated variable levels of detectable SARS-CoV-2 transcript, which were not statistically significant between variants (FIG. 3C). Notably, supernatants from infected kidney organoids reveal significantly decreased levels of replicating virus from both the Alpha and Gamma strains, compared to one of the originally isolated WA1 strains (FIG. 3D). Additionally, LDH release from infected kidney organoids was not significantly different between viral strains, nor heightened compared to mock infected controls (FIG. 11 and FIG. 18). Together, this data from organoids and clinical AKI prevalence suggest that VOCs do not have an increased toll on kidney health (see also FIG. 14).

### ACE2 is the Primary Viral Entry Pathway for SARS-CoV-2 Infection

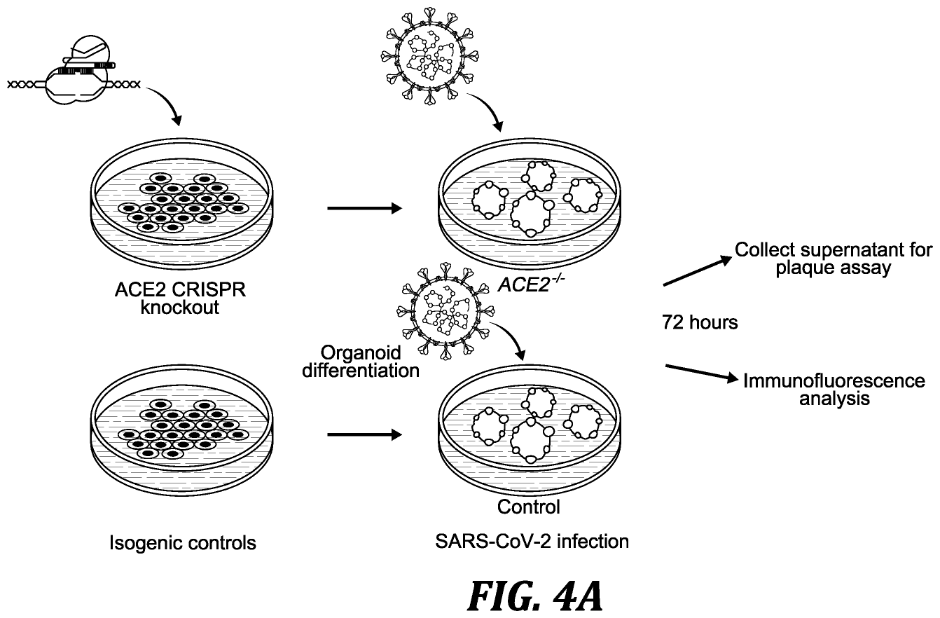

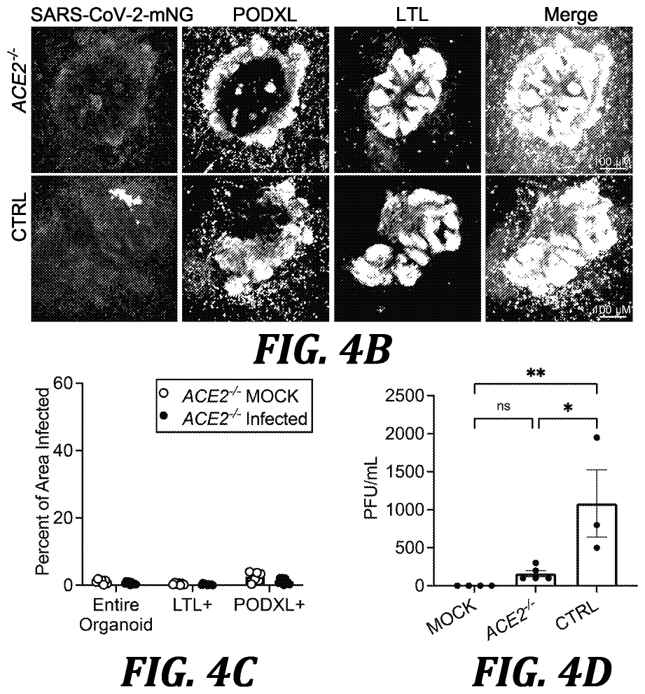

Susceptibility of kidney organoids to SARS-CoV-2 infection is thought to depend upon expression of ACE2, but genetic proof of this is lacking. To assess this, genetically modified ACE2^{-/-} stem cell lines were utilized, compared to ACE2^{+/+} controls (FIG. 4A). When exposed to SARS-CoV-2-mNG, however, ACE2^{-/-} organoids did not express detectable mNG-fluorescence, in contrast to ACE2^{+/+} controls (FIG. 4B and FIG. 4C; see also FIG. 19A and FIG. 19B). Supernatants from ACE2^{-/-} organoid cultures showed 85% fewer viral particles than supernatants from ACE2^{30 /+} controls and were not significantly different in viral production from MOCK infected controls (FIG. 4D).

### Therapeutics Reduce SARS-CoV-2 Infection and Replication in Kidney Organoids

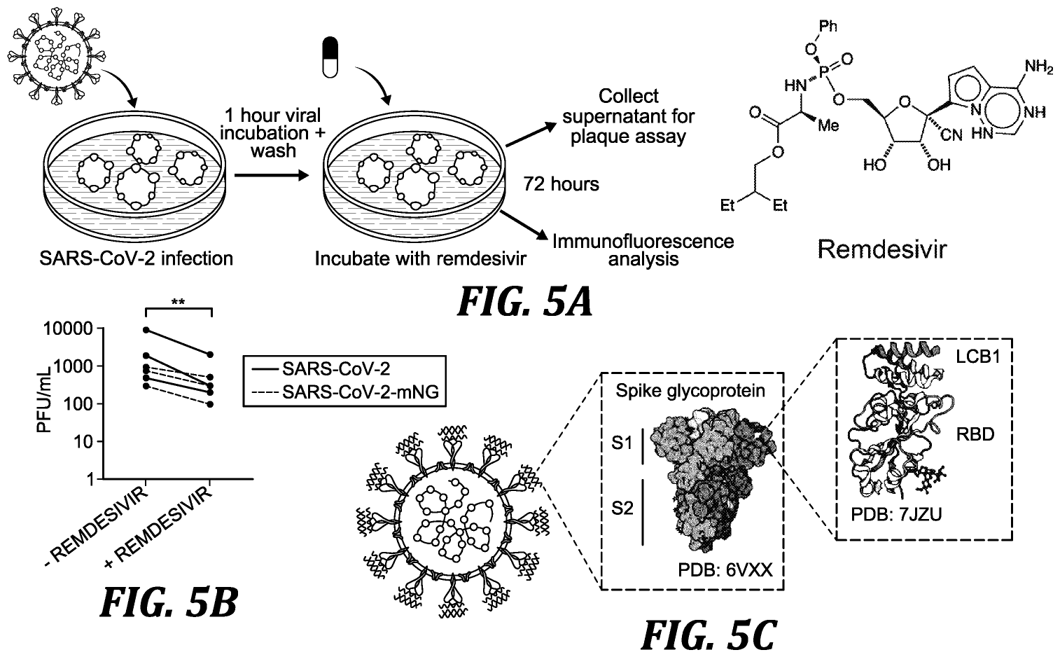

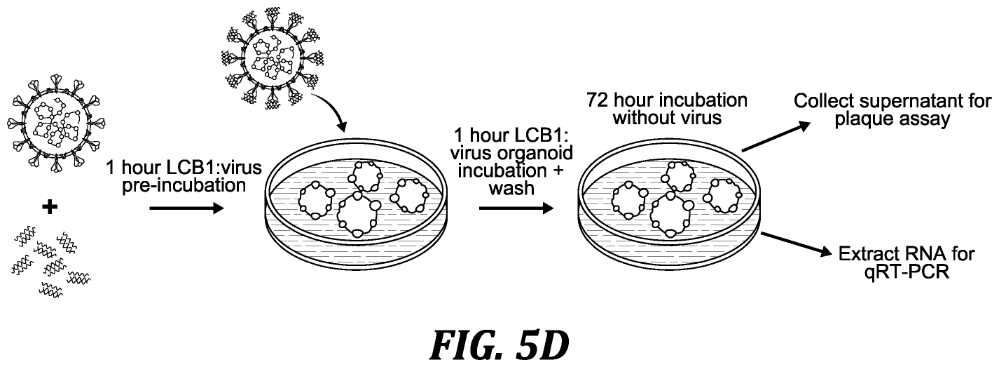

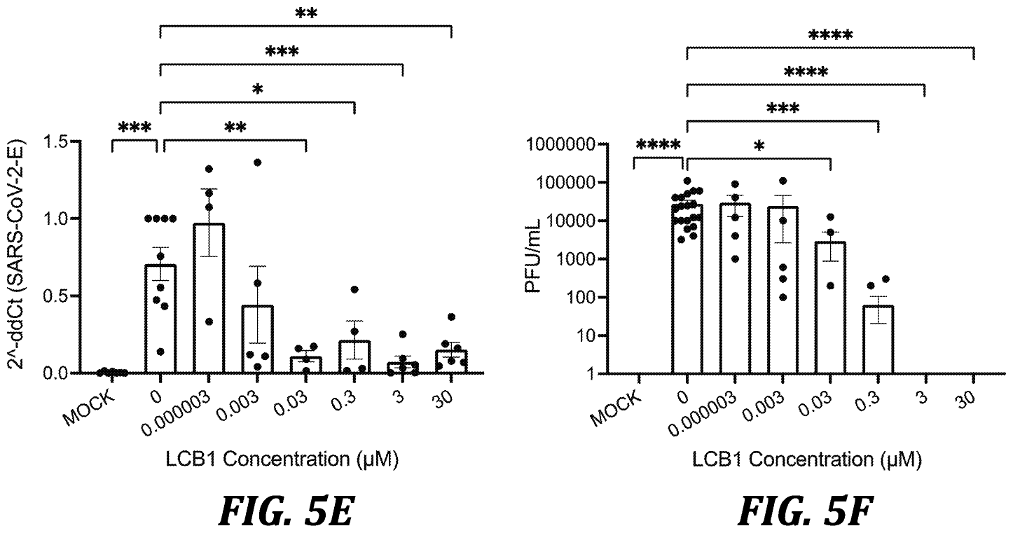

To investigate the efficacy of remdesivir, kidney organoids were infected with SARS-CoV-2 or SARS-CoV-2-mNG, and then treated with a 2 μM dose of remdesivir immediately after infection (FIG. 5A). Supernatants from treated and untreated organoids were collected, revealing 71.4%±18% reduced replicated virus in the remdesivir treated organoids (FIG. 5B). These data suggest that remdesivir significantly reduces viral replication of infected kidney organoids and supports short term safety of remdesivir treatment in kidney cells at the efficacious dose, while cautioning that overdose of the drug may be counterproductive for the kidneys.

While remdesivir appears to show efficacy in vitro, it is not efficacious in vivo in lowering mortality or reducing infection in COVID-19 patients, necessitating the development of alternatives. The de novo designed protein, LCB1, was specifically designed to bind the receptor binding domain of SARS-CoV-2's spike protein at picomolar concentrations and has been estimated to have six-fold greater potency than monoclonal antibodies but has not yet been tested for efficacy in renal tissues (FIG. 5C). To assess whether LCB1 can block SARS-CoV-2 infection and replication in a kidney-relevant system, 0 μM-30 μM of LCB1 was preincubated with an MOI of 10 of SARS-CoV-2 for 1 hour, and the LCB1:virus mixture was added to kidney organoids (FIG. 5D). qRT-PCR analysis of RNA extracted from infected organoids demonstrated an LCB1 dose-dependent decrease of detectable SARS-CoV-2 transcript, with significantly different levels at 0.03 μM and higher (FIG. 5E). Supernatants collected from infected organoids were assessed via plaque assay, revealing a dose-dependent decrease in viral particles, starting at ≥0.03 μM, with complete abrogation at the 3 μM and higher doses (FIG. 5F). Thus, LCB1 can efficiently block SARS-CoV-2 infection at levels sufficient to prevent viral replication in human kidney organoids.

### Adeno-Associated Viruses do not Efficiently Transduce Kidney Organoids

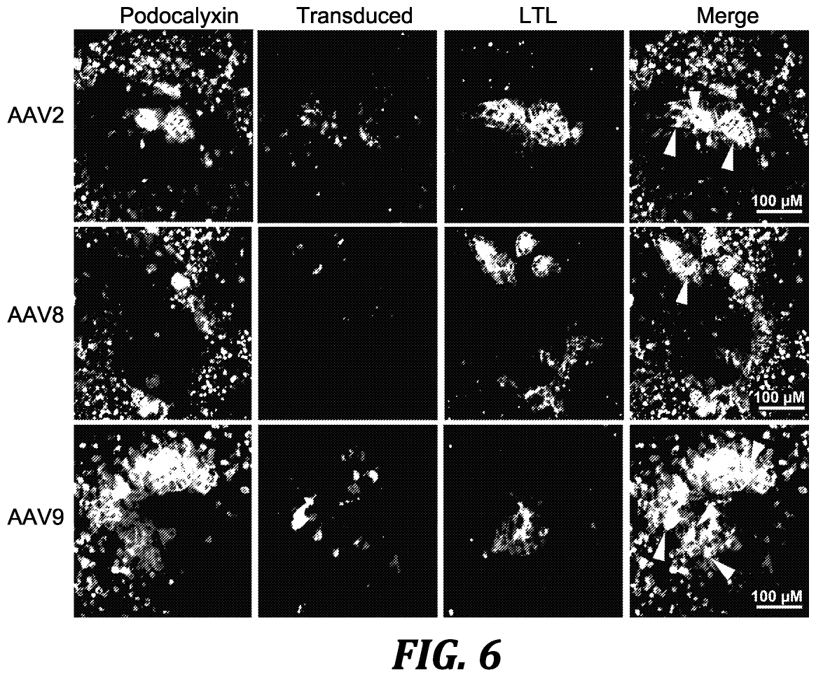

To assess whether AAVs were able to infect kidney organoids, organoids were transduced with an AAV vector of serotype 2, 6, 8, or 9 with an mCherry reporter at Day 10 of differentiation. Organoids were grown to maturity prior to staining with nephron markers podocalyxin and LTL (FIG. 6). Only AAVs 2, 8, and 9 had detectable infection in the kidney, but at very low levels of efficiency. The AAVs were able to colocalize with both podocalyxin and LTL.

VSVG-Lentivirus does not Efficiently Transduce Mature Kidney Organoids

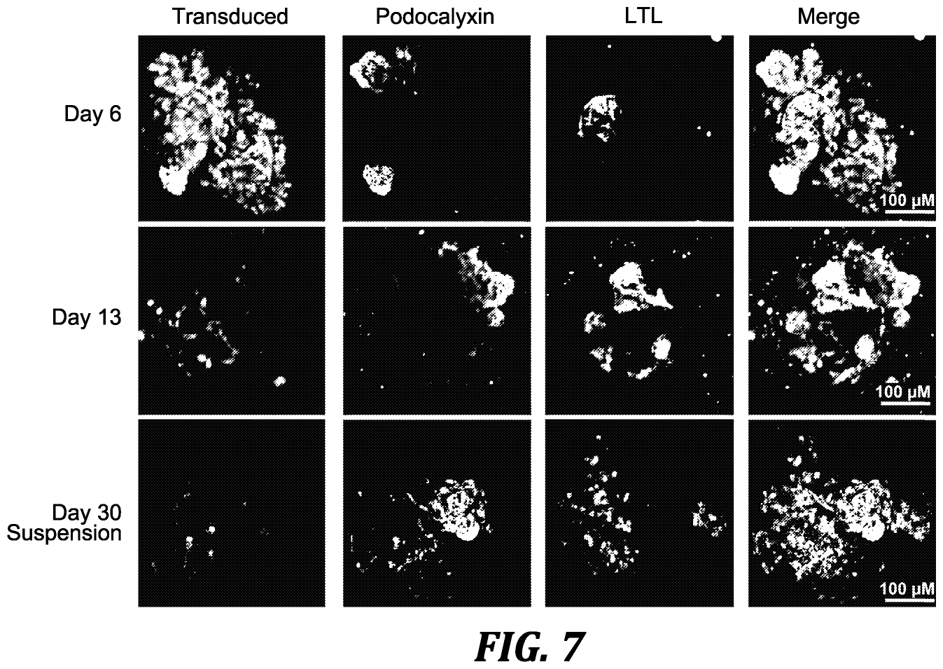

To assess whether VSVG-pseudotyped lentiviruses were able to productively infect kidney organoids, organoids were transduced with a lentivirus with a GFP reporter at different stages of maturity, Day 6, Day 13, and fully matured in suspension culture at Day 30 to assess efficiency and tropism of the virus (FIG. 7). Transducing organoids at early stages of differentiation yielded high levels of GFP throughout the organoid colocalizing with both podocytes and proximal tubules. Transducing at Day 13 of the organoid differentiation resulted in targeting only to the kidney stromal epithelium, and not to either the podocytes or proximal tubules. Transduction of fully mature kidney organoids yielded next to no efficiency at transducing any part of the organoid, potentially indicating a kidney epithelial barrier to viral entry as structures mature.

Together, these data indicate that SARS-CoV-2 is the most efficient viral vector for targeting the mature kidney epithelium, particularly the proximal tubules, and that gene therapy vector delivery strategies can utilize SARS-CoV-2 envelopes and/or spike proteins for proximal tubule kidney specificity.

Gene Expression Changes by Organoids Because of SARS-CoV-2 Infection Correlate with Proteomic Markers Associated with Diagnosis of COVID-19

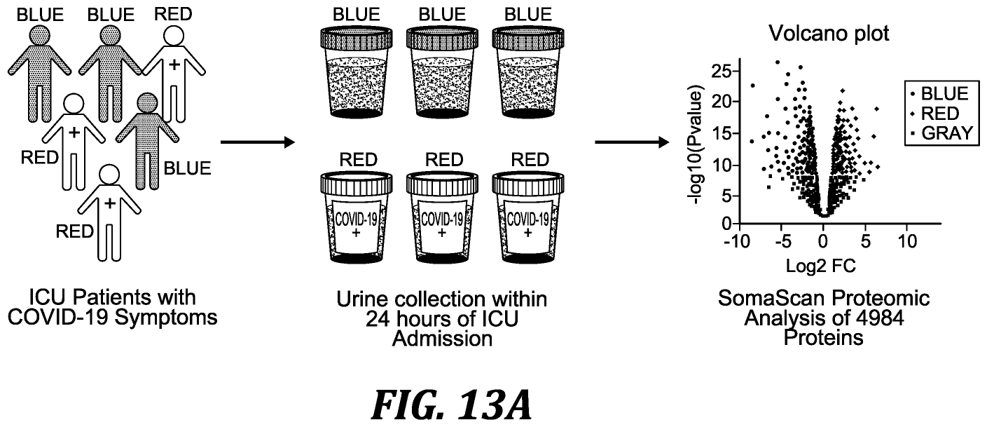

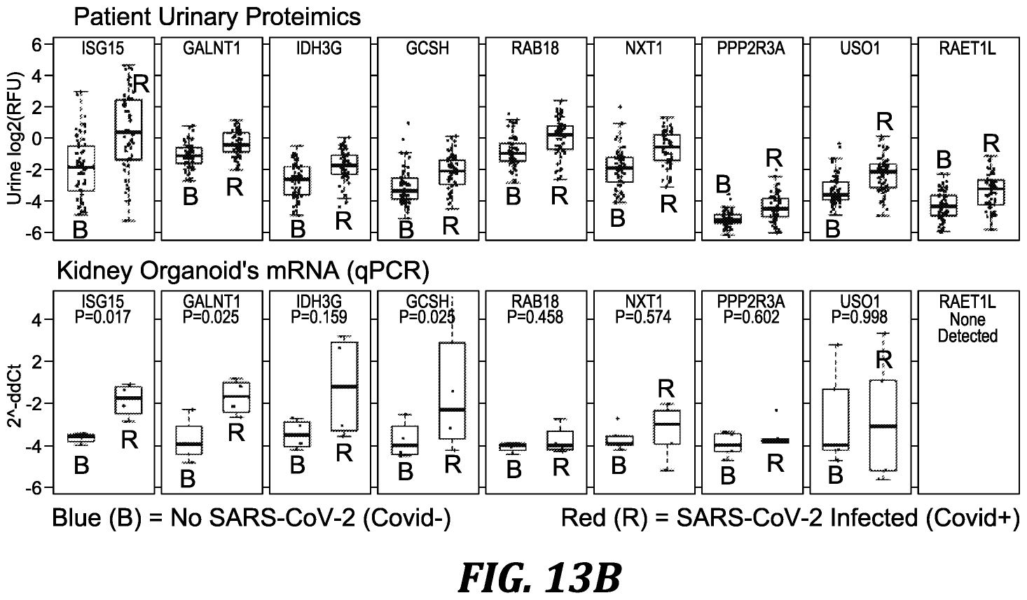

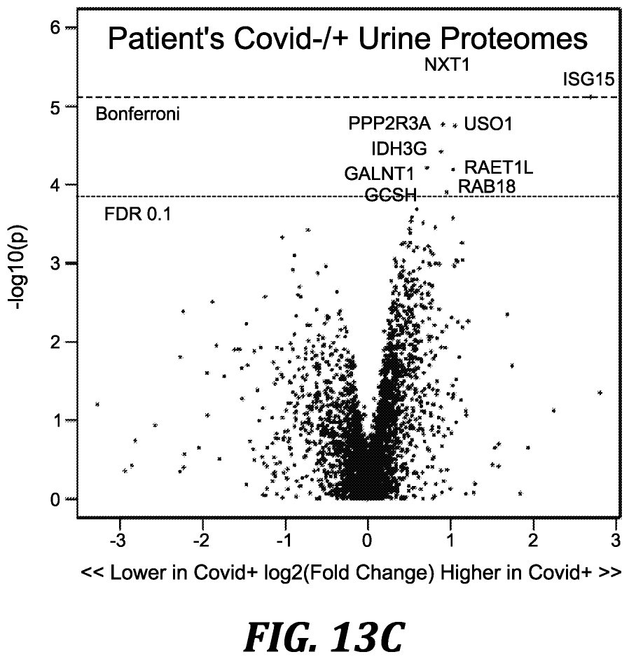

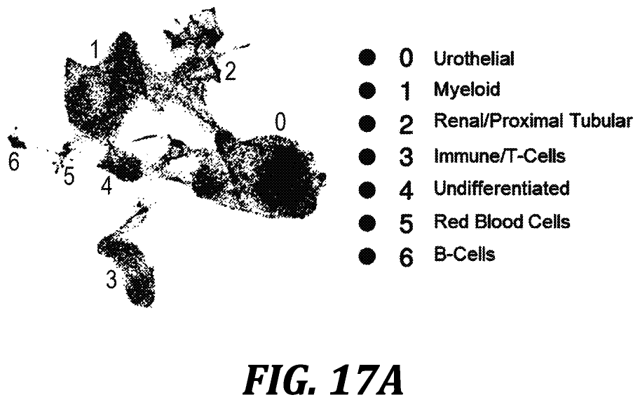

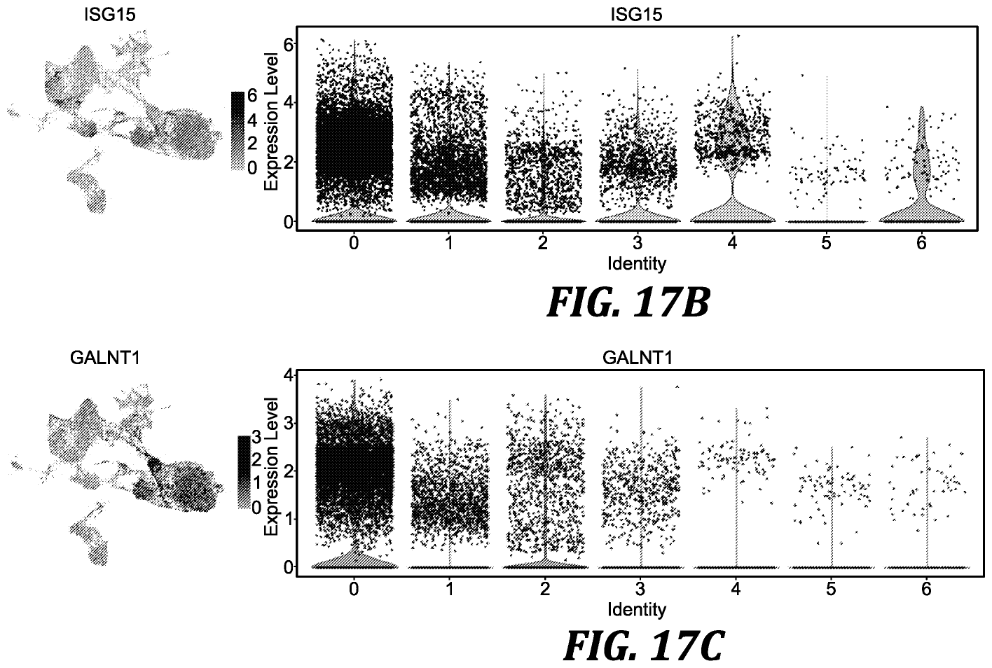

To determine whether the responses of the kidney organoid model system to infection with SARS-CoV-2 virus are representative of or consistent with responses of patients who have COVID-19, experiments were conducted to compare proteomics of patients' urine (SomaScan) with mRNA markers of kidney organoids infected with SARS-CoV-2 (qPCR). As shown at FIG. 13A, a workflow for a proteomic analysis of urine from patients who are either COVID-19 negative (BLUE) or COVID-19 positive (RED) was followed to collect urine within 24 hours of ICU admission, and a Volcano plot of proteomics data (FIG. 13C) was produced to correlate COVID-19 diagnosis status (positive/RED or negative/BLUE) with urine protein markers. It was found that mRNA transcripts that correspond with proteins that are altered in COVID-19 positive patients compared to COVID-19 negative patients were similarly modulated (e.g., upregulated, or downregulated) in kidney organoid cultures (FIG. 13B). To evaluate which cell types or tissues contributed to these features, ISG15 and GALNT1 expression levels were determined with scRNAseq in urothelial, myeloid, renal/proximal tubular, immune/T-cells, undifferentiated, red blood cells, and B-cells (FIG. 17A, FIG. 17B, FIG. 17C).

### Representative Image Analysis Workflow for a SARS-CoV-2-mNG Infected WTC11 Organoid

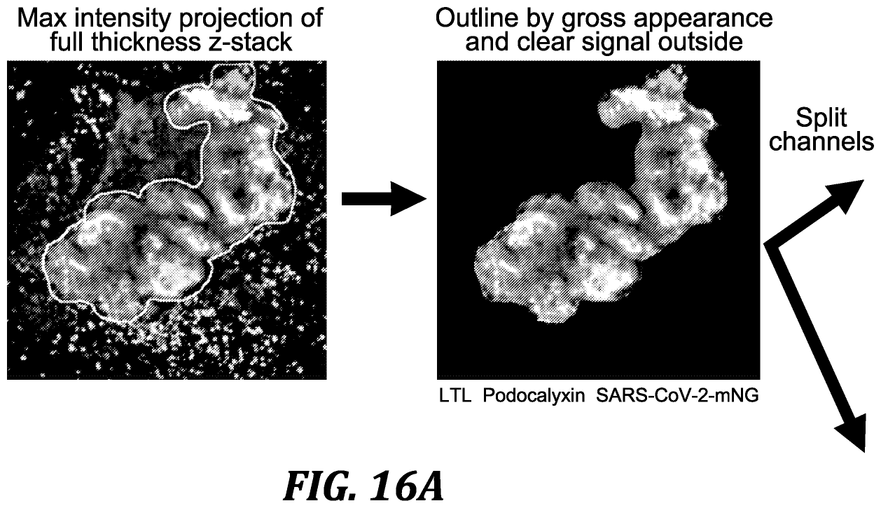

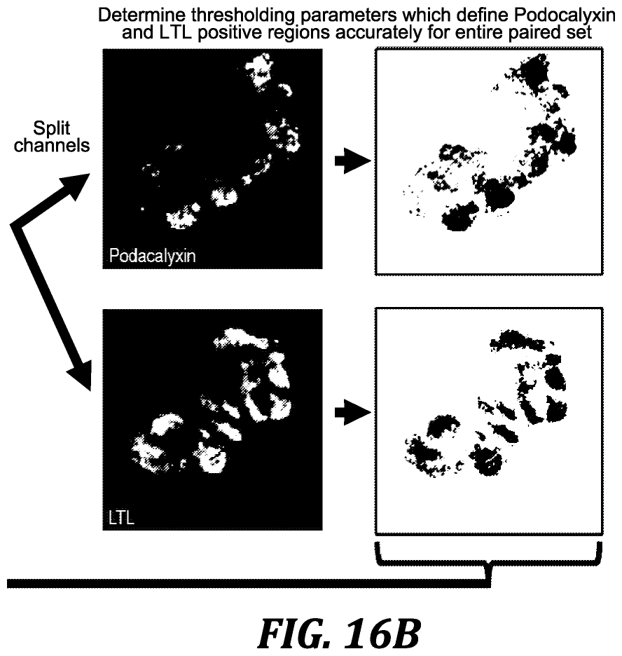

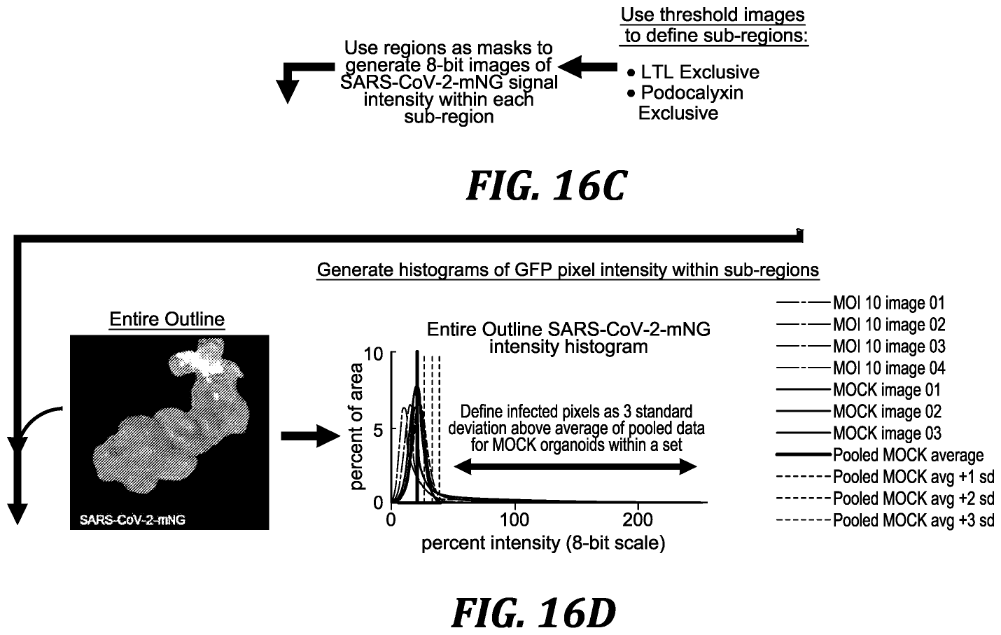

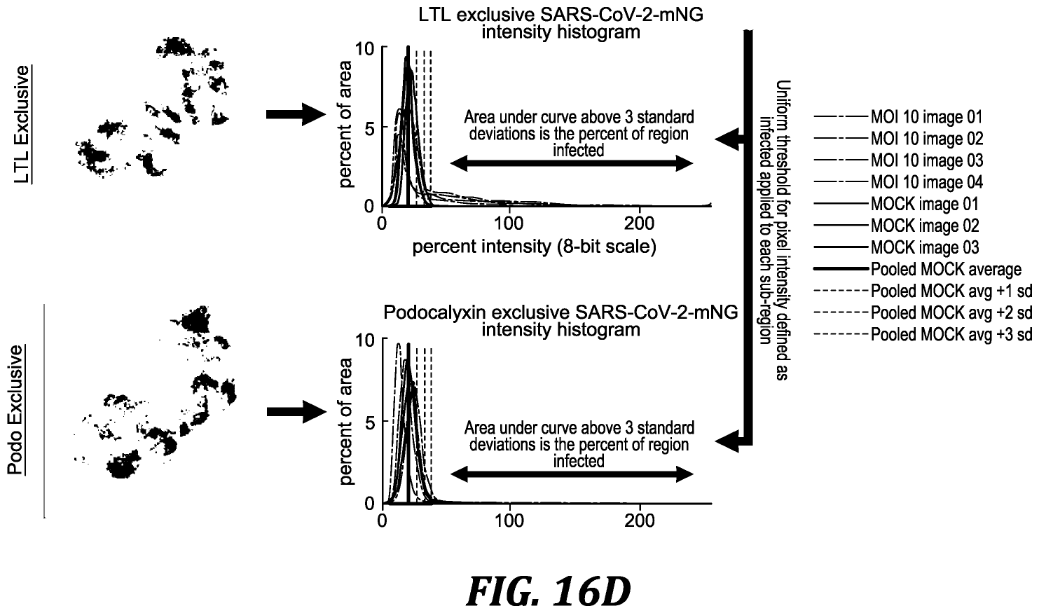

Confocal Z-stacks are converted to maximum intensity projections; the organoid is then manually outlined with outside signal cleared to restrict analysis to signal within the organoid body (FIG. 16A). Representative multi-channel composite images are split into individual channels for podocalyxin and LTL and the user determines threshold parameters for each channel which accurately define podocalyxin- and LTL-positive regions. Uniform thresholding parameters for each channel are applied to all Mock and infected organoid images within a paired set (FIG. 16B). Binary thresholded images are used to define areas that are exclusively positive for either podocalyxin or LTL. Regions with vertically adjacent distinct cell types artifactually appear co-stained in projection and are excluded from analysis. Podocalyxin- and LTL-exclusive binary images are used as masks to generate images of SARS-CoV-2-mNG pixel intensity within each defined sub-region (FIG. 16C). Histograms of pixel intensity are generated for each organoid within a Mock and infected paired set. Histograms are normalized based on size of organoid or region to convert raw pixel counts to percent of area, such that each organoid contributes equally to statistical analysis. Normalized histograms of pixel intensity of the entire organoid outline for all Mock organoids within a particular set are pooled together and the average and standard deviation for the pooled data is quantified. Pixels that are greater than 3 standard deviations above average pixel intensity for the pooled Mock data are defined as infected. By definition, for a perfect normal distribution Mock organoids are expected to have 0.15% of pixels defined as infected on average. The pixel intensity threshold for infection determined from the entire organoid outline is then uniformly applied to both the podocalyxin- and LTL-exclusive regions for each Mock and infected organoid image within a set to determine the percent infection of each area for all organoids (FIG. 16D).

### Example 2: Pharmaceutical Prevention of Interferon-Mediated Nephropathy and APOL1 Upregulation in Kidney Organoids

As a result of experiments that involved treatment of human kidney organoids with SARS-CoV-2 viruses and/or variants and/or components thereof, it was observed that a substantial upregulation of the interferon gamma (IFN-γ) pathway occurred in the treated organoids. The cytokine IFN-γ is an essential mediator of the innate and adaptive immune response. Chronic inflammatory infections induce persistent IFN-γ upregulation causing expression of numerous IFN-stimulated genes. IFN-γ is commonly used to induce apolipoprotein L-1 (APOL1) expression in cell models to study APOL1-associated nephropathy; however, the effect of IFN-γ on nephron structures itself remains poorly studied. In this example, IFN-γ-induced APOL1 expression, localization, and pharmacological inhibition are characterized in kidney nephron organoids. It is further demonstrated that prolonged IFN-γ exposure itself results in a pronounced loss of endothelial networks and disorganization of tubular structures. In contrast, podocyte integrity appears unaffected as demonstrated with time-lapse imaging of organoids expressing GFP-tagged podocalyxin and staining for junctional components. Isolated primary adult kidney endothelial cells show a similar sensitivity to IFN-γ treatment. These results help establish kidney organoids as a model for studies on chronic inflammatory nephrological conditions and characterize important side effects of IFN-γ when inducing endogenous expression of APOL1.

Several patterns of kidney damage related to interferon treatment have been observed in case studies. These include endothelial damage (e.g., Thrombotic microangiopathy, Thrombotic thrombocytopenia purpura, Atypical haemolytic uremic syndrome), glomerular damage (e.g., Focal segmental glomerulosclerosis, Membranoproliferative glomerulonephritis, Minimal-change disease, Membranous nephropathy), and tubular/interstitial damage (e.g., Acute tubular necrosis, Thrombotic thrombocytopenia purpura).

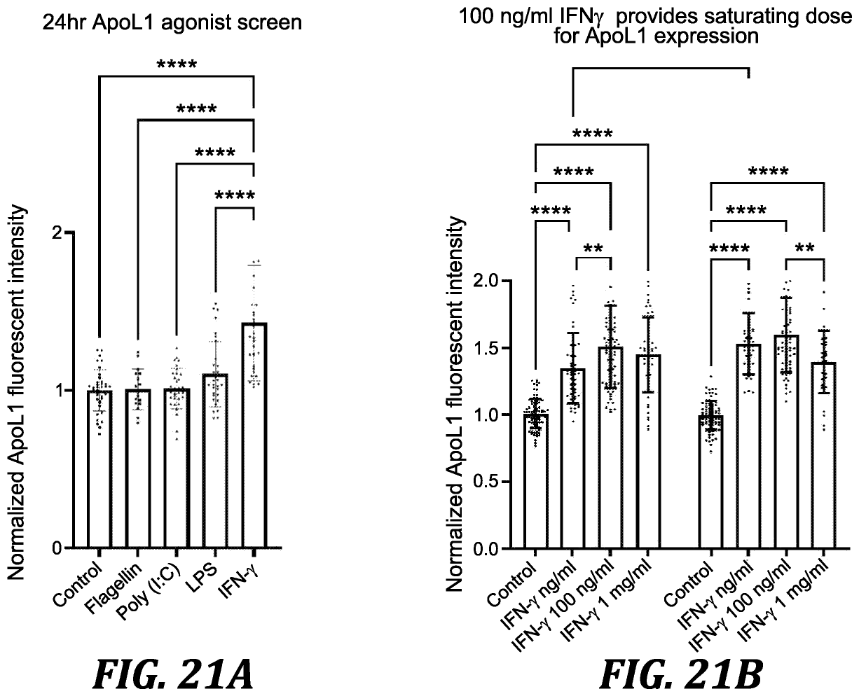

Experiments were performed to determine APOL1 localization in normal human kidney and stimulation in kidney organoids. It was found that APOL1 expression in normal healthy human tissue appears most strongly in glomeruli but is also present in tubules (results from confocal immunofluorescence microscopy of control; markers: APOL1, DAPI, Non-specific 2° staining). In addition, results from representative images of organoids fluorescently labeled for APOL1 and quantified for fluorescent intensity show that 100 ng/mL IFNγ treatment provides a saturating dose for APOL1 expression (results from confocal immunofluorescence microscopy of control vs. IFN-γ 100 ng/ml (24 hr); markers: LTL, Podocalyxin, APOL1, DAPI). Further, it was found that APOL1 sub-cellular localization appears to be primarily membrane associated (results from confocal immunofluorescence microscopy of control; markers: Podocalyxin, LTL, DAPI, CD31, APOL1). As shown at FIG. 21A, a 24-hour APOL1 agonist screen revealed IFN-γ to be a strong agonist for APOL1 expression, and titration of IFN-γ revealed a dose-responsive activation of APOL1 expression (FIG. 21B).

Next, it was determined whether pharmacological inhibition and reversion of APOL1 expression with JAK ½ inhibitors was feasible with the organoid model system. Based on canonical type-2 interferon signaling and APOL1 induction (FIG. 22D), several JAK ½ inhibitors (to inhibit APOL1 expression or cause reversion of APOL1 expression) were tested for their ability to impact expression of several genes (FIG. 22A). Single-cell RNA sequencing (scRNAseq) results for control+vehicle, control+1 day IFN treatment, and IFN treatment in combination with JAK ½ inhibitors as shown for inhibition of APOL1 expression (FIG. 22B), as well as scRNAseq results for control+vehicle, control+1 day IFN treatment, control+3 day IFN treatment, and IFN treatment in combination with JAK ½ inhibitors as shown for reversion of APOL1 expression (FIG. 22C) revealed that pharmacological inhibition and reversion of APOL1 expression with JAK ½ inhibitors is feasible with the organoid model system.

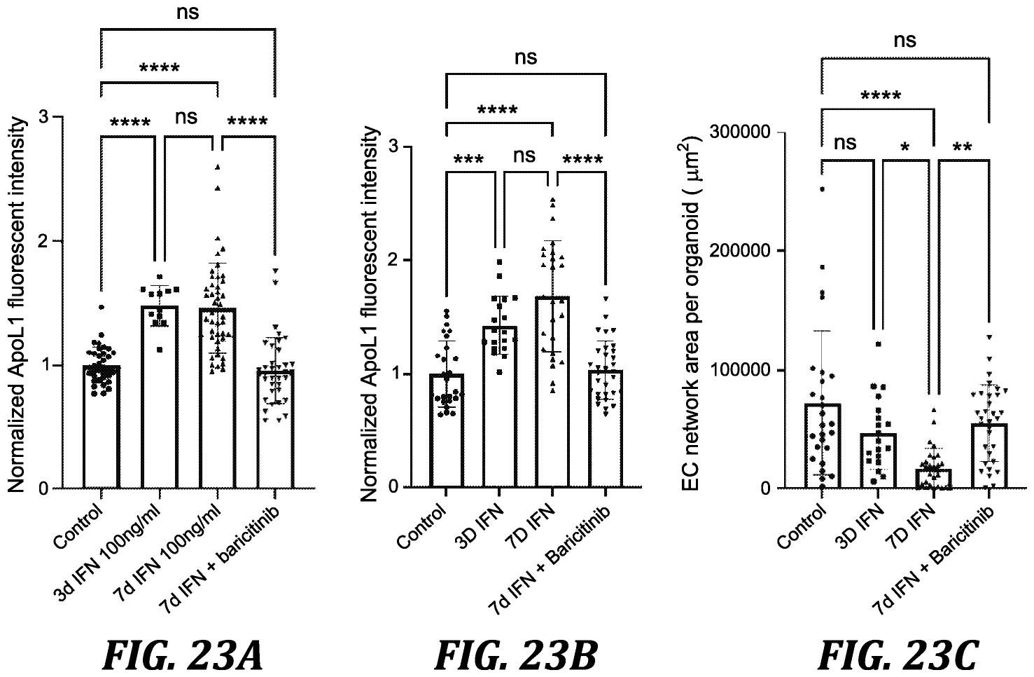

It was also found that prolonged treatment with IFN-γ resulted in pronounced loss of endothelial networks and tubular structures and tubule networks become disorganized (results from confocal immunofluorescence microscopy of control vs. 7d IFN vs. 7d IFN+Baricitinib; markers: CD31, APOL1, GFP-PODXL, DAPI; results from light microscopy and confocal immunofluorescence microscopy of control vs. 7d IFN vs IFN+bari; markers: PODXL, LTL, APOL1, DAPI). As such, it was next determined whether JAK ½ inhibition prevents IFN-γ-induced loss of endothelial networks and disorganization of tubular structures. It was found that treatment with baricitinib prevents APOL1 upregulation in the organoid body (FIG. 23A), prevents APOL1 upregulation in EC vessels (FIG. 23B), and prevents vascular regression that occurs with prolonged IFN-γ treatment (FIG. 23C). As a result, it was concluded that JAK ½ inhibition prevents IFN-γ-induced loss of endothelial networks and disorganization of tubular structures.

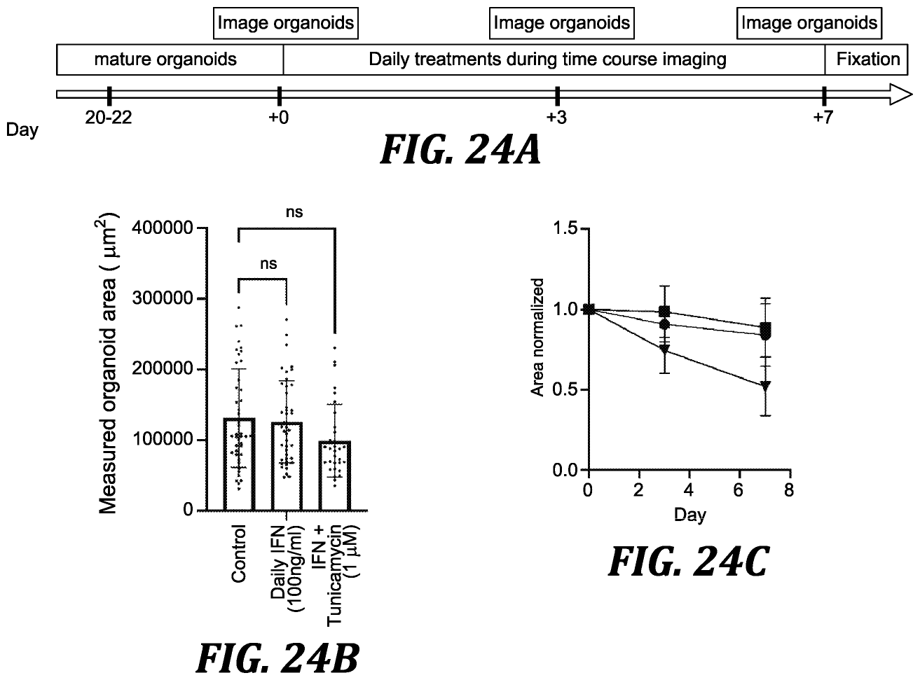

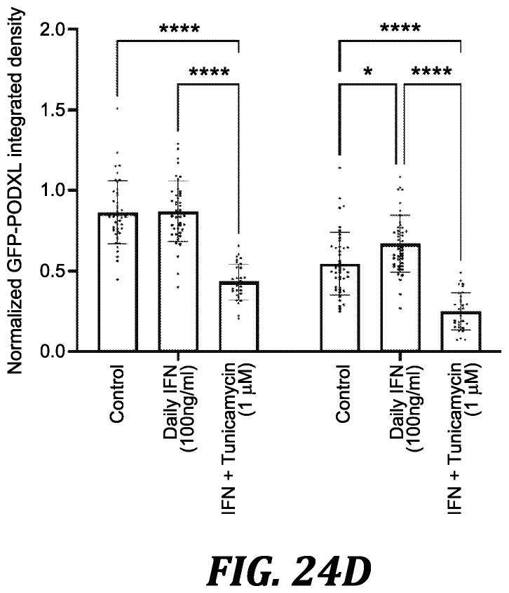

It was determined whether podocytes are damaged by prolonged IFN-γ treatment. As shown at FIG. 24A, an example time course for prolonged IFN-γ treatment involves regular imaging of organoids during treatment of mature organoids with IFN-γ prior to fixation. It was found that IFN-γ treatment does not result in enhanced loss of GFP-PODXL (FIG. 24B), and that only tunicamycin treated organoids shrink in size (FIG. 24C and FIG. 24D). Further, podocyte junctional tracks appear intact with 7d IFN-γ treatment (results from confocal immunofluorescence microscopy of control versus 7d IFN-γ treatment; markers: ZO-1, Synaptopodin, ZO-1+Synaptopodin, APOL1, and DAPI). Based on these observations, it was concluded that podocytes appear largely undamaged by prolonged IFN-γ treatment.

While illustrative embodiments have been illustrated and described, it will be appreciated that various changes can be made therein without departing from the spirit and scope of the invention.

1. A method for culturing a human kidney organoid as a model system for characterization of a pathogenesis, the method comprising:
generating a culture that comprises the human kidney organoid and a human kidney organoid maintenance medium for the culturing of the human kidney organoid;
contacting the culture with a viral preparation that comprises a severe acute respiratory syndrome coronavirus 2 (SARS-CoV-2) virus, a variant thereof, or a component thereof; and
determining whether one or more cells or cell types of the human kidney organoid are infected by the viral preparation and produce a SARS-CoV-2 infected human kidney organoid.

2. The method of claim 1, wherein one or more cells or cell types of the human kidney organoid are angiotensin converting enzyme 2 (ACE2) positive.

3. The method of claim 1, further comprising:
modulating an ACE2 gene in one or more cells or cell types of the human kidney organoid to produce one or more ACE2 modulated cells or cell types; and
determining, after contacting the culture with the viral preparation, whether the one or more ACE2 modulated cells or cell types are differentially infected by the viral preparation relative to one or more cells or cell types that are not ACE2 modulated cells or cell types.

4. The method of claim 1, further comprising:
knocking out an ACE2 gene in one or more cells or cell types of the human kidney organoid to produce one or more ACE2 negative cells or cell types; and
determining, after contacting the culture with the viral preparation, whether the one or more ACE2 negative cells or cell types are not infected by the viral preparation.

5. The method of claim 1, wherein one or more cells or cell types of the human kidney organoid have a genetic modification that is associated with polycystic kidney disease (PKD genotype).

6. The method of claim 1, further comprising:
creating a PKD genotype in one or more cells or cell types of the human kidney organoid to produce one or more PKD genotype cells or cell types; and
determining, after contacting the culture with the viral preparation, whether the one or more PKD genotype cells or cell types are infected by the viral preparation.

7. The method of claim 1, further comprising:
knocking out a PKD2 gene in one or more cells or cell types of the human kidney organoid to produce one or more PKD2 negative cells or cell types; and
determining, after contacting the culture with the viral preparation, whether the one or more PKD2 negative cells or cell types are infected by the viral preparation.

8. The method of claim 1, wherein the human kidney organoid comprises one or more genome edited cells comprising one or more genomic edits.

9. The method of claim 8, wherein the one or more genomic edits comprises:
a modulation, a knockdown, or a knockout of an ACE2 gene;
a modulation, a knockdown, or a knockout of one or more genes that are associated with polycystic kidney disease (PKD gene), wherein the PKD gene comprises a polycystin-1 gene (PKD1), a polycystin-2 gene (PKD2) and/or a polycystic kidney and hepatic disease 1 gene (PKHD1); and/or
a knock in of a pathogenic APOL1 gene.

10. The method of claim 8, wherein the characterization comprises an evaluation of a tropism or a response of one or more cells or cell types that comprise the human kidney organoid to a pathological agent.

11. (canceled)

12. The method of claim 1, wherein the maintenance medium comprises an anti-pathological agent for at least potential interference with a pathological process.

13. The method of claim 12, wherein the pathological process comprises infection with a virus and/or replication of the virus and the anti-pathological agent comprises an antiviral agent.

14. (canceled)

15. The method of claim 1, wherein the determining whether one or more cells or cell types of the human kidney organoid are infected comprises evaluating whether the one or more cells or cell types express a gene product that comprises a fluorescent marker.

16. The method of claim 1, further comprising:
contacting the culture with a molecule for at least partial inhibition and/or reversion of APOL1 expression by the human kidney organoid.

17. (canceled)

18. The method of claim 16, wherein the molecule is a JAK ½ inhibitor.

19. (canceled)

20. The method of claim 1, wherein the SARS-CoV-2 variant is a WA1 variant, an Alpha variant, a Beta variant, a Gamma variant, or a Delta variant.

21. A model system for characterization of a pathogenesis, the model system comprising:
a SARS-CoV-2 infected human kidney organoid; and
a human kidney organoid maintenance medium for culture of the SARS-CoV-2 infected human kidney organoid.

22-36. (canceled)

37. A method for characterizing a pathophysiology, the method comprising:
culturing a culture that comprises a SARS-CoV-2 infected human kidney organoid and a human kidney organoid maintenance medium for maintenance of the human kidney organoid;
contacting the culture with a pathological agent; and
characterizing a response of the culture to the pathological agent.

38-50. (canceled)

51. A method to virally transduce a human kidney cell, the method comprising:
culturing a human kidney organoid in a human kidney organoid maintenance medium;
contacting the culture with an agent that comprises a virus, a variant of the virus, or a component of the virus; and
characterizing a response of the culture to the agent;
wherein the response of the culture informs development of viral transduction of the human kidney cell.

52-57. (canceled)

58. A method for gene therapy of a mature kidney epithelium of a human kidney, the method comprising:
contacting the mature kidney epithelium with a SARS-CoV-2 virus vector, a SARS-CoV-2 variant virus vector, and/or a virus vector that comprises a component of an envelope and/or a spike protein of the SARS-CoV-2 virus vector or the SARS-CoV-2 variant virus vector.

59-62. (canceled)
